SEQUENCE LISTING (22 sequences)

SEQ ID NO: 1 - protein, 769 aa, Homo sapiens
  REGION 1..769: note = Description of sequence: Uniprot P05107-1 amino acid sequence
MLGLRPPLLALVGLLSLGCVLSQECTKFKVSSCRECIESGPGCTWCQKLNFTGPGDPDSIRCDTRPQLLMRGCAADDIMDPTSLAETQEDHNGGQKQLSPQKVTLYLRPGQAAAFNVTFRRAKGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVQLIKNAYNKLSSRVFLDHNALPDTLKVTYDSFCSNGVTHRNQPRGDCDGVQINVPITFQVKVTATECIQEQSFVIRALGFTDIVTVQVLPQCECRCRDQSRDRSLCHGKGFLECGICRCDTGYIGKNCECQTQGRSSQELEGSCRKDNNSIICSGLGDCVCGQCLCHTSDVPGKLIYGQYCECDTINCERYNGQVCGGPGRGLCFCGKCRCHPGFEGSACQCERTTEGCLNPRRVECSGRGRCRCNVCECHSGYQLPLCQECPGCPSPCGKYISCAECLKFEKGPFGKNCSAACPGLQLSNNPVKGRTCKERDSEGCWVAYTLEQQDGMDRYLIYVDESRECVAGPNIAAIVGGTVAGIVLIGILLLVIWKALIHLSDLREYRRFEKEKLKSQWNNDNPLFKSATTTVMNPKFAES

SEQ ID NO: 2 - protein, 114 aa, synthetic construct
  REGION 1..114: note = Description of sequence: Antibody D3-7065 heavy chain variable region;
  REGION 29..34: note = Complementarity-determining region
  REGION 50..59: note = Complementarity-determining region
  REGION 99..101: note = Complementarity-determining region
EVQLVESGGGLVQPGGSLRLSCAASGFTISYYYMHWVRQAPGKGLEWVASISSSSGYTYYADSVKGRFTISADTSKNTAYLQMNSLRAEDTAVYYCARGAMDYWGQGTLVTVSS

SEQ ID NO: 3 - protein, 111 aa, synthetic construct
  REGION 1..111: note = Description of sequence: Antibody D3-7065 light chain variable region
  REGION 29..33: note = Complementarity-determining region
  REGION 51..57: note = Complementarity-determining region
  REGION 92..100: note = Complementarity-determining region
DDIQMTQSPSSLSASVGDRVTITCRASQSVSSAVAWYQQKPGKAPKLLIYSASSLYSGVPSRFSGSRSGTDFTLTISSLQPEDFATYYCQQFSSGSWAPITFGQGTKVEIK

SEQ ID NO: 4 - protein, 229 aa, synthetic construct
  REGION 1..229: note = Description of sequence: C1-7065-H-L orientation C1 linker
EVQLVESGGGLVQPGGSLRLSCAASGFTISYYYMHWVRQAPGKGLEWVASISSSSGYTYYADSVKGRFTISADTSKNTAYLQMNSLRAEDTAVYYCARGAMDYWGQGTLVTVSSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQFSSGSWAPITFGQGTKVEIK

SEQ ID NO: 5 - protein, 234 aa, synthetic construct
  REGION 1..234: note = Description of sequence: C2-7065-H-L orientation C2 linker
EVQLVESGGGLVQPGGSLRLSCAASGFTISYYYMHWVRQAPGKGLEWVASISSSSGYTYYADSVKGRFTISADTSKNTAYLQMNSLRAEDTAVYYCARGAMDYWGQGTLVTVSSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQFSSGSWAPITFGQGTKVEIK

SEQ ID NO: 6 - protein, 239 aa, synthetic construct
  REGION 1..239: note = Description of sequence: C3-7065-H-L orientation C3 linker
EVQLVESGGGLVQPGGSLRLSCAASGFTISYYYMHWVRQAPGKGLEWVASISSSSGYTYYADSVKGRFTISADTSKNTAYLQMNSLRAEDTAVYYCARGAMDYWGQGTLVTVSSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQFSSGSWAPITFGQGTKVEIK

SEQ ID NO: 7 - protein, 244 aa, synthetic construct
  REGION 1..244: note = Description of sequence: C4-7065-H-L orientation C4 linker
EVQLVESGGGLVQPGGSLRLSCAASGFTISYYYMHWVRQAPGKGLEWVASISSSSGYTYYADSVKGRFTISADTSKNTAYLQMNSLRAEDTAVYYCARGAMDYWGQGTLVTVSSGGGGSGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQFSSGSWAPITFGQGTKVEIK

SEQ ID NO: 8 - protein, 229 aa, synthetic construct
  REGION 1..229: note = Description of sequence: D1-7065-L-H orientation D1 linker
DIQMTQSPSSLSASVGDRVTITCRASQSVSSAVAWYQQKPGKAPKLLIYSASSLYSGVPSRFSGSRSGTDFTLTISSLQPEDFATYYCQQFSSGSWAPITFGQGTKVEIKGGGGSEVQLVESGGGLVQPGGSLRLSCAASGFTISYYYMHWVRQAPGKGLEWVASISSSSGYTYYADSVKGRFTISADTSKNTAYLQMNSLRAEDTAVYYCARGAMDYWGQGTLVTVSS

SEQ ID NO: 9 - protein, 234 aa, synthetic construct
  REGION 1..234: note = Description of sequence: D2-7065-L-H orientation D2 linker
DIQMTQSPSSLSASVGDRVTITCRASQSVSSAVAWYQQKPGKAPKLLIYSASSLYSGVPSRFSGSRSGTDFTLTISSLQPEDFATYYCQQFSSGSWAPITFGQGTKVEIKGGGGSGGGGSEVQLVESGGGLVQPGGSLRLSCAASGFTISYYYMHWVRQAPGKGLEWVASISSSSGYTYYADSVKGRFTISADTSKNTAYLQMNSLRAEDTAVYYCARGAMDYWGQGTLVTVSS

SEQ ID NO: 10 - protein, 239 aa, synthetic construct
  REGION 1..239: note = Description of sequence: D3-7065-L-H orientation D3 linker
DIQMTQSPSSLSASVGDRVTITCRASQSVSSAVAWYQQKPGKAPKLLIYSASSLYSGVPSRFSGSRSGTDFTLTISSLQPEDFATYYCQQFSSGSWAPITFGQGTKVEIKGGGGSGGGGSGGGGSEVQLVESGGGLVQPGGSLRLSCAASGFTISYYYMHWVRQAPGKGLEWVASISSSSGYTYYADSVKGRFTISADTSKNTAYLQMNSLRAEDTAVYYCARGAMDYWGQGTLVTVSS

SEQ ID NO: 11 - protein, 244 aa, synthetic construct
  REGION 1..244: note = Description of sequence: D4-7065-L-H orientation D4 linker
DIQMTQSPSSLSASVGDRVTITCRASQSVSSAVAWYQQKPGKAPKLLIYSASSLYSGVPSRFSGSRSGTDFTLTISSLQPEDFATYYCQQFSSGSWAPITFGQGTKVEIKGGGGSGGGGSGGGGSGGGGSEVQLVESGGGLVQPGGSLRLSCAASGFTISYYYMHWVRQAPGKGLEWVASISSSSGYTYYADSVKGRFTISADTSKNTAYLQMNSLRAEDTAVYYCARGAMDYWGQGTLVTVSS

SEQ ID NO: 12 - protein, 20 aa, synthetic construct
  REGION 1..20: note = Description of sequence: (Gly-Gly-Gly-Gly-Ser)n Linker
  REGION 1..20: note = Sequence GGGGS may be repeated for --non-limiting-- example four times
  VARIANT 6..20: note = Amino Acids may be absent
GGGGSGGGGSGGGGSGGGGS

SEQ ID NO: 13 - protein, 6 aa, synthetic construct
  REGION 1..6: note = Description of sequence: Antibody component: HCDR1
ISYYYM

SEQ ID NO: 14 - protein, 10 aa, synthetic construct
  REGION 1..10: note = Description of sequence: Antibody component: HCDR2
SISSSSGYTY

SEQ ID NO: 15 - skipped

SEQ ID NO: 16 - protein, 5 aa, synthetic construct
  REGION 1..5: note = Description of sequence: Antibody component: LCDR1
SVSSA

SEQ ID NO: 17 - protein, 7 aa, synthetic construct
  REGION 1..7: note = Description of sequence: Antibody component: LCDR2
SASSLYS

SEQ ID NO: 18 - protein, 9 aa, synthetic construct
  REGION 1..9: note = Description of sequence: Antibody component: LCDR3
FSSGSWAPI

SEQ ID NO: 19 - protein, 5 aa, synthetic construct
  REGION 1..5: note = Description of sequence: Linker for C1 and D1
GGGGS

SEQ ID NO: 20 - protein, 10 aa, synthetic construct
  REGION 1..10: note = Description of sequence: Linker for C2 and D2
GGGGSGGGGS

SEQ ID NO: 21 - protein, 15 aa, synthetic construct
  REGION 1..15: note = Description of sequence: Linker for C3 and D3
GGGGSGGGGSGGGGS

SEQ ID NO: 22 - protein, 20 aa, synthetic construct
  REGION 1..20: note = Description of sequence: Linker for C4 and D4
GGGGSGGGGSGGGGSGGGGS

(19) United States
(12) Patent Application Publication
(10) Pub. No.: US 2026/0137781 A1
(43) Pub. Date: May 21, 2026

(54) Antibodies and Immunotherapies That Target the Active Conformation of Integrin Beta 2

(71) Applicant: The Regents of the University of California, Oakland, CA (US)

(72) Inventors: Arun Wiita, Oakland, CA (US); Kamal Mandal, Oakland, CA (US)

(21) Appl. No.: 18/973,891

(22) Filed: Dec. 9, 2024

Related U.S. Application Data
(63) Continuation of application No. PCT/CA2023/050780, filed on Jun. 7, 2023.
(60) Provisional application No. 63/350,299, filed on Jun. 8, 2022.

(51) Int. Cl. A61K 40/11; A61K 35/17; A61K 40/15; A61K 40/31; A61K 40/42; A61K 40/50; A61K 47/68; A61P 35/02; C07K 16/28

(52) U.S. Cl. CPC A61K 40/11; A61K 35/17; A61K 40/15; A61K 40/31; A61K 40/42; A61K 40/50; A61K 47/6851; A61P 35/02; C07K 16/2845; A61K 2239/48; C07K 2317/31; C07K 2317/565; C07K 2317/622

(57) ABSTRACT

Provided herein are antibodies to active integrin beta-2 and compositions comprising such antibodies for the treatment of a cancer, e.g., acute myeloid leukemia.

### CROSS-REFERENCE TO RELATED APPLICATIONS

This application is a continuation of PCT/CA2023/050780, international filing date Jun. 7, 2023, which claims benefit of priority to U.S. Provisional Patent Application No. 63/350,299, filed Jun. 8, 2022, each of which is incorporated by referenced herein for all purposes.

### STATEMENT AS TO RIGHTS TO INVENTIONS MADE UNDER FEDERALLY SPONSORED RESEARCH AND DEVELOPMENT

This invention was made with government support under grant R21 CA263229 awarded by the National Institutes of Health. The government has certain rights in the invention.

### SEQUENCE LISTING IN ELECTRONIC FORMAT

The contents of the electronic sequence listing (081906-1477176-250010US_ST26.xml; Size: 34,346 bytes; and Date of Creation: Jul. 24, 2025) is herein incorporated by reference in its entirety.

### BACKGROUND OF THE INVENTION

Development of safe and effective immunotherapy for acute myeloid leukemia (AML). Although targets, such as CD123, CD33, CD70 and CLEC12A, have been identified, no immunotherapies are approved to date and none are appropriate for a wide range of AML patients, given the heterogeneity and complexity of the disease.

### BRIEF SUMMARY OF ASPECTS OF THE DISCLOSURE

We employed a strategy to target conformation-specific cancer antigens. Such antigens are not possible to identify using gene expression/RNA-seq or traditional proteomics approaches. We therefore employed a structural proteomics methodology, termed cross-linking mass spectrometry (XL-MS), to address the problem. We integrated cell surface enrichment proteomics with XL-MS and identified the active conformation of Integrin β2 (Itgβ2) as an AML target. Antibodies against activated Itgβ2 were engineered using a phage display platform. We then reconstructed the antibodies into chimeric antigen receptor-T (CAR-T) and demonstrated specific cytotoxicity against AML tumor models.

In one aspect, provided herein an antibody that specifically binds to activated integrin beta-2 (Itgβ2), wherein the antibody comprises an Itgβ2 binding domain comprising: a heavy chain variable region (VH) comprising an HCDR1 sequence comprising ISYYYM, an HCDR2 sequence comprising SISSSSGYTY; and an HCDR3 sequence comprising GAM; and a light chain variable region (VL) comprising an LCDR1 sequence comprising SVSSA, an LCDR2 sequence comprising SASSLYS; and an LCDR3 sequence comprising FSSGSWAPI. In some embodiments, the V_{H} comprises an amino acid sequence having at least 95% identity to SEQ ID NO:2 and/or the V_{L} comprises an amino acid sequence having at least 95% identity to SEQ ID NO:3. In some embodiments, the V_{H} comprises amino acid sequence SEQ ID NO:2 and/or the V_{L} comprises amino acid sequence SEQ ID NO:3. In some embodiments, the antibody is a single chain Fv (scFv). In some embodiments, the V_{H} region of the scFv is N-terminal to the V_{L} region. In some embodiments, the V_{L} region of the scFv is N-terminal to the V_{H} region. In some embodiments, the V_{H} and the V_{L} regions of the scFV are separated by a flexible linker. In some embodiments, the flexible linker comprises Gly-Ser. In some embodiments, the flexible linker comprises one or more Gly4Ser sequences. In some embodiments, the scFv comprises the amino acid sequence of any one of SEQ ID NOS:4-11. In some embodiments, the scFv comprises the amino acid of SEQ ID NO:10 or SEQ ID NO: 11.

In a further aspect, the disclosure provides a chimeric antigen receptor (CAR) comprising an antigen binding domain, a transmembrane domain, and an intracellular domain comprising a costimulatory domain and/or a primary signaling domain, wherein the antigen binding domain comprises an antibody as described herein, e.g., in the preceding paragraph. In some embodiments, the antigen binding domain comprises the antibody comprising the sequence of any one of SEQ ID NOS: 4-11. In some embodiments, the antigen binding domain comprises the sequence of SEQ ID NO: 10 or SEQ ID NO:11. In addition, the disclosure provides a chimeric antigen receptor (CAR) comprising an antigen binding domain, a transmembrane domain, and a cytoplasmic signaling domain comprising a costimulatory domain and/or a primary signaling domain, wherein the antigen binding domain comprises an antibody as described herein, e.g., in the preceding paragraph. In some embodiments, the antigen binding domain comprises the sequence of any one of SEQ ID NOS:4-11. In some embodiments, the antigen binding domain comprises the sequence of SEQ ID NO:10 or SEQ ID NO:11.

In a further aspect, the disclosure provides an immune effector cell comprising a CAR as described herein, e.g., a CAR as described in the preceding paragraph. In some embodiments, the immune effector cell is a T lymphocyte or a natural killer (NK) cell.

In a further aspect, the disclosure provides a method of treating a cancer that comprises cancer cells that express active Itgβ2, the method comprising administering a plurality of immune effector cells, as described herein, e.g., in the preceding paragraph, to a subject that has the cancer. In some embodiments, the plurality of immune effector cells comprises allogeneic cells. In some embodiments, the plurality of immune effector cells comprises autologous cells. In some embodiments, the cancer is acute myeloid leukemia.

In a further aspect, the disclosure provides a polynucleotide encoding a CAR as described herein, e.g., in this section of the application. In some embodiments, the disclosure provides a vector, e.g., a retroviral vector such as a self-inactivating lentiviral vector, comprising the polynucleotide. In addition, the disclosure provides an immune effector cell comprising a polynucleotide or vector as described herein, e.g., in the current paragraph. In some embodiments, the immune effector cell is a T lymphocyte or NK cell. Further, the disclosure provides a host cell comprising a polynucleotide as described herein, e.g., in the present paragraph. In some embodiments, the host cell is a T lymphocyte or NK cell. In a further embodiment, the disclosure provides a nucleic acid encoding a V_{H} and/or a V_{L} region as described herein, such as a V_{H} comprising an HCDR1 sequence comprising ISYYYM, an HCDR2 sequence comprising SISSSSGYTY; and an HCDR3 sequence comprising GAM; and a V_{L} comprising an LCDR1 sequence comprising SVSSA, an LCDR2 sequence comprising SASSLYS; and an LCDR3 sequence comprising FSSGSWAPI. In some embodiments, the V_{H} comprises an amino acid sequence having at least 95% identity to SEQ ID NO:2 and/or the V_{L} comprises an amino acid sequence having at least 95% identity to SEQ ID NO:3. In some embodiments, the V_{H} comprises amino acid sequence SEQ ID NO:2 and/or the V_{L} comprises amino acid sequence SEQ ID NO:3.

In some embodiments, the disclosure provides a nucleic acid encoding an antibody of any one of SEQ ID NOS:4-11, and an expression vector and/or host cell comprising such an antibody.

### BRIEF DESCRIPTION OF THE DRAWINGS

FIG. 1A-D provides a schematic diagram of surfaceomics.

FIG. 2A-E illustrates that activated Itgβ2 is a conformationally selective antigen of AML. A) Cross-linked peptides mapped on crystal structure of Itgal/Itgβ2. 2B-E: Flow cytometry analysis showing activated Itgβ2 B) presence on AML cell lines C) absence on HSPCs D) presence on primary AML cells and E) presence only in the hematopoietic system and not elsewhere in the body.

FIG. 3A-D. Development of antibody against activated Itgβ2. A) Schematic diagram showing phage display library platform B) Triage showing screening and identification of antibody specific for activated Itgβ2 C) Represtative plot showing kD determination of 7065 Fab against Itgal/Itgβ2 D) Flow cytometry based screening of antibodies specific for activated Itgβ2.

FIG. 4A-E. Development and optimization of CAR-T against AML, henceforth to be referred as aItgβ2 CAR-T A) Schematic diagram of CAR-T construct B) Luciferase based cytotoxicity of aItgβ2 CAR-T design (varying by 1-4 linker tandem repeats between heavy and light chain) against Nomo1 cell line C) Incucyte-based proliferation assay showing the potency of the aItgβ2 CAR-T cells against Nomo1 D) Flowcytometry data showing successful generation of Itgβ2-KO version of Nomo 1 E) Cytotoxicity of aItgβ2 CAR-T against Nomo 1 WT and its Itgβ2-KO version to demonstrate its specificity.

FIG. 5A-I Toxicity assessment of aItgβ2 CAR-T. A and C) Cytotoxicity assay showing specificity of aItgβ2 CAR-T against activated activated T cells B) Flow cytometry analysis showing activated T cells population being partially positive for activated Itgβ2 D) Clonogenic assay showing non-toxicity of aItgβ2 CAR-T against HSPCs E) Flow cytometry analysis showing non-toxicity of aItgβ2 CAR-T against T cells and B cells. F) Flow diagram showing strategy of HIS mice generation G) HIS mice data showing non-toxicity of aItgβ2 CAR-T against myeloid cells (CD14+). H) Bar graph showing compromised abundance of human cells (CD45+) post CAR-T treatment in case of CD33, but not Itgβ2 I) CBC profiling of mice treated with CAR-T.

FIG. 6A-E. A) Survival curve showing prolonged lifespan of mice treated with aItgβ2 CAR-T compared to that of empty CAR-T. Mice were infused with AML PDX 5 days before CAR-T treatment. B) Flow cytometry analysis of mice blood draw showing absence of tumor cells with aItgβ2 CAR-T treatment. C) Ultrasonography of spleen showed enlarged spleen in empty CAR-T compared to aItgβ2 CAR-T treated mice. D) Bioluminescence imaging showed reduced tumor burden in aItgβ2 CAR-T mice. Mice were infused with luciferase labelled Nomo 1 (AML cell line) 5 days before CAR-T treatment E) Quantitative analysis of bioluminescence signal obtained in (D).

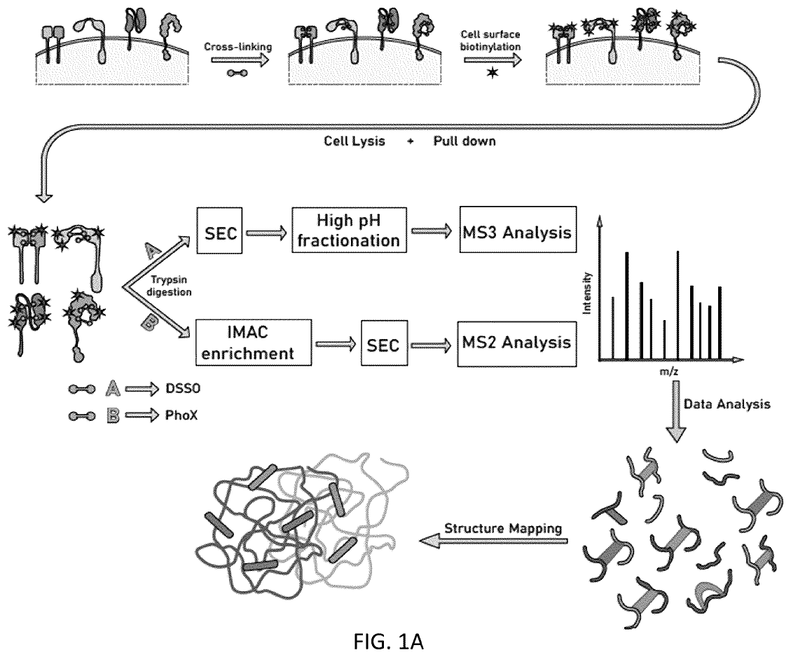

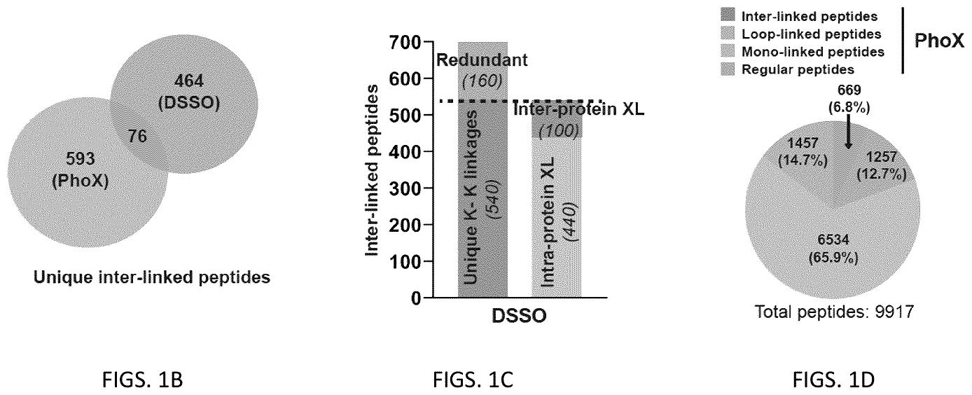

### DETAILED DESCRIPTION OF THE DISCLOSURE

### Terminology

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “about” as used herein refers to the usual error range for the respective value readily known to the skilled person in this technical field. For example, for K_{D} and IC₅₀ values ±20%, ±10%, or ±5%, are within the intended meaning of the recited value.

The term “integrin beta 2” or “itgβ2”, also known as CD18, LAD, LCAMB, LFA-1, MAC-1, MF17, MFI7, or integrin subunit beta 2, as used here refers to a polypeptide that is encoded by a ITGB2 gene (chr21:44,885,949-44,931,989 (GRCH38/hg38), cytogenetically localized to human chromosome 21q22.3 by HGNC, Entrez Gene, and Ensembl (genomic coordinates (GRCh38/hg38 assembly December 2013) and plays a role in cell adhesion, cell-surface-mediated sequencing, and immune responses. An illustrative human Itgβ2 protein sequence encoded by a human ITGB2 gene, P05107-1, is available under Uniprot number P05107 and is provided as SEQ ID NO:1. Itgβ2 can bind to a number of alpha chains and thus can from multiple heterodimers, but also exists in soluble, ligand binding forms. Deficiencies in Itgb2 expression can lead to adhesion defects in circulating white blood cells in humans, reducing the immune system's ability to fight off foreign invaders. Illustrative Itgb2 heterodimers include, e.g., integrin ITGAL/ITGB2, which is a receptor for ICAM1, ICAM2, ICAM3 and ICAM4, and is also a receptor for the secreted form of ubiquitin-like protein ISG15; integrins ITGAM/ITGB2 and ITGAX/ITGB2, which are receptors for the iC3b fragment of the third complement component and for fibrinogen; integrin ITGAX/ITGB2, which recognizes the sequence G-P-R in fibrinogen alpha-chain, Integrin ITGAM/ITGB2, which recognizes P1 and P2 peptides of fibrinogen gamma chain and is also a receptor for factor X; and integrin ITGAD/ITGB2, which is a receptor for ICAM3 and VCAM1.

The terms “anti-Itgβ2 antibody,” “Itgβ2 specific antibody,” “Itgβ2 antibody,” and “anti-Itgβ2” are used synonymously herein to refer to an antibody that specifically binds to Itgβ2. An illustrative human Itgβ2 sequence is provided in SEQ ID NO:1.

An “anti-Itgβ2 binding domain” as used herein refers to an antigen binding domain comprising a V_{H} and a V_{L} region of an anti-Itgβ2 antibody as described herein, which antigen binding domain binds to active conformation Itgβ2.

An an anti-Itgβ2 antibody of the present disclosure binds to an active form of Itgβ2. An active state of Itgβ2 is an extended-open conformation (see, e.g., Nishida et al, Immunity 25:583-94, 2006; Li et al, EMBO J. 36:629-45, 2017). The active conformation (extended-open) has a 4,000-fold increase in ligand affinity compared to the other two states (bent-closed, inactive; and extended-closed (intermediate) (Li et al., 2017, supra). Integrin activation takes place upon cell stimulation through various cell surface receptors. Cell stimulation triggers an inside-out signaling pathway that ultimately recruits cytoplasmic factors such as talin and kindlin to the NPxY motifs of the cytoplasmic tail of the integrin's beta-chain, which causes the cytoplasmic tails of the integrin subunits to separate and switches the integrin to the active (extended-open) conformation.

The term “antibody” refers to a polypeptide comprising a framework region encoded by an immunoglobulin gene, or fragments thereof, that specifically binds and recognizes an antigen, e.g., the activated form of Itgβ2. Typically, the “variable region” contains the antigen-binding region of the antibody (or its functional equivalent) and is important in specificity and affinity of binding. The term “antibody” as used herein thus encompasses antigen binding fragments, e.g., an antigen binding domain, or other antigen binding fragment. Antigen binding fragments may be produced by modification of whole antibodies, or produced using recombinant DNA methodologies (e.g., single chain Fv formats).

An illustrative immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

As used herein, “V-region” refers to an antibody variable region domain comprising the segments of Framework 1, CDR1, Framework 2, CDR2, and Framework 3, including CDR3 and Framework 4, which segments are added to the V-segment as a consequence of rearrangement of V-region genes during B-cell differentiation.

As used herein, “complementarity-determining region (CDR)” refers to the three hypervariable regions that interrupt the four “framework” regions of a variable domain. The CDRs are the primary contributors to binding to an epitope of an antigen. The CDRs of are referred to as CDR1, CDR2, and CDR3, numbered sequentially starting from the N-terminus.

The amino acid sequences of the CDRs and framework regions can be determined using various well known definitions in the art, e.g., Kabat, Chothia, international ImMunoGeneTics database (IMGT), and AbM (see, e.g., Johnson et al., supra; Chothia & Lesk, 1987, Canonical structures for the hypervariable regions of immunoglobulins. J. Mol. Biol. 196, 901-917; Chothia C. et al., 1989, Conformations of immunoglobulin hypervariable regions. Nature 342, 877-883; Chothia C. et al., 1992, structural repertoire of the human VH segments J. Mol. Biol. 227, 799-817; Al-Lazikani et al., J. Mol. Biol 1997, 273(4)). Definitions of antigen combining sites are also described in the following: Ruiz et al., IMGT, the international ImMunoGeneTics database. Nucleic Acids Res., 28, 219-221 (2000); and Lefranc, M.-P. IMGT, the international ImMunoGeneTics database. Nucleic Acids Res. Jan 1; 29(1):207-9 (2001); MacCallum et al, Antibody-antigen interactions: Contact analysis and binding site topography, J. Mol. Biol., 262 (5), 732-745 (1996); and Martin et al, Proc. Natl Acad. Sci. USA, 86, 9268-9272 (1989); Martin, et al, Methods Enzymol., 203, 121-153, (1991); Pedersen et al, Immunomethods, 1, 126, (1992); and Rees et al, In Sternberg M. J. E. (ed.), Protein Structure Prediction. Oxford University Press, Oxford, 141-172 1996). Reference to CDRs as determined by Kabat numbering are based, for example, on Kabat et al., Sequences of Proteins of Immunological Interest, 5th Ed. Public Health Service, National Institute of Health, Bethesda, MD (1991)). Chothia CDRs are determined as defined by Chothia (see, e.g., Chothia and Lesk J. Mol. Biol. 196:901-917 (1987)).

An “isotype” is a class of antibodies defined by the heavy chain constant region. Antibodies described herein can be of any isotype of isotype class. Immunoglobulin genes include the kappa, lambda, alpha, gamma, delta, epsilon, and mu constant region genes. Light chains are classified as either kappa or lambda. Heavy chains are classified as gamma, mu, alpha, delta, or epsilon, which in turn define the isotype classes, IgG, IgM, IgA, IgD and IgE, respectively. In some embodiments, the IgG is an IgG1, IgG2, IgG3 or IgG4.

Antibodies can exist as intact immunoglobulins or as any of a number of well-characterized fragments that include specific antigen-binding activity. Such fragments can be produced by digestion with various peptidases. Pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology.

Antibodies or antigen-binding molecules of the invention further includes one or more immunoglobulin chains that are chemically conjugated to, or expressed as, fusion proteins with other proteins. It also includes bispecific antibody. A bispecific or bifunctional antibody is an artificial hybrid antibody having two different heavy/light chain pairs and two different binding sites. Other antigen-binding fragments or antibody portions of the invention include bivalent scFv (diabody), bispecific scFv antibodies where the antibody molecule recognizes two different epitopes, single binding domains (dAbs), and minibodies. The term “antibody” additionally encompasses bispecific and multispecific antibodies as well as any other monovalent, bivalent, or multivalent antibody format.

The various antibodies or antigen-binding fragments described herein can be produced by enzymatic or chemical modification of the intact antibodies, or synthesized de novo using recombinant DNA methodologies (e.g., single chain Fv), or identified using yeast or phage display libraries (see, e.g., McCafferty et al., Nature 348:552-554, 1990; Boder, et al (2000) Proc. Natl. Acad. Sci. U.S.A 97:10701). For example, minibodies can be generated using methods described in the art, e.g., Vaughan and Sollazzo, Comb Chem High Throughput Screen. 4:417-30 2001. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148, 1547-1553 (1992). Single chain antibodies can be identified using phage display libraries, yeast display, or ribosome display libraries, gene shuffled libraries. Such libraries can be constructed from synthetic, semi-synthetic or native and immunocompetent sources.

A “monoclonal antibody” refers to a clonal preparation of antibodies with a single binding specificity and affinity for a given epitope on an antigen.

A “chimeric antibody” is an antibody molecule in which (a) the constant region, or a portion thereof, is altered, replaced or exchanged so that the antigen binding site (variable region, CDR, or portion thereof) is linked to a constant region of a different or altered class, effector function and/or species, or an entirely different molecule which confers new properties to the chimeric antibody (e.g., an enzyme, toxin, hormone, growth factor, drug, etc.); or (b) the variable region, or a portion thereof, is altered, replaced or exchanged with a variable region having a different or altered antigen specificity (e.g., CDR and framework regions from different species).

A “humanized” antibody is an antibody that retains the reactivity of a non-human antibody while being less immunogenic in humans. This can be achieved, for instance, by retaining the non-human CDR regions and replacing the remaining parts of the antibody with their human counterparts. In one embodiment, some, most or all of the amino acids outside the CDR domains are replaced with amino acids corresponding to the human immunoglobulin germline, while amino acids within one or more CDR regions are unchanged. In some embodiments, one or more CDR residues may be altered, e.g., to provide a sequence closer to germline or to replace a residue that may impede production.

The term “specifically bind” refers to a molecule (e.g., antibody or antibody fragment) that binds to a target with at least 2-fold greater affinity than non-target compounds, e.g., at least 4-fold, 5-fold, 6-fold, 7-fold, 8-fold, 9-fold, 10-fold, 20-fold, 25-fold, 50-fold, or 100-fold greater affinity. For example, an antibody that specifically binds Itgβ2, e.g., active Itgβ2, typically bind to Itgβ2, e.g., active Itgβ2, with at least a 2-fold greater affinity than a non-Itgβ2 target, or in the case of an antibody that specifically binds active Itgβ2, an inactive form of Itgβ2. In some embodiments, an antibody binds toactive Itgβ2 with a K_{D} that is at least 100-fold greater than its affinity inactive Itgβ2.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The term “valency” as used herein refers to the number of different binding sites of an antibody for an antigen. A monovalent antibody comprises one binding site for an antigen. A multivalent antibody comprises multiple binding sites.

The words “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

A “flexible linker” as described herein refers to an amino acid sequence that joins domains to provide a certain degree of movement or interaction. Such linkers are generally composed of small, non-polar (e.g. Gly) or polar (e.g. Ser or Thr) amino acids, but may also comprise polar amino acids such as Lys and Glu, e.g., to improve solubility. The small size of these amino acids provides flexibility, and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties. In some embodiments, flexible linkers are primarily composed of stretches of Gly and Ser residues (“GS” linker). An example of the most widely used flexible linker has the sequence of (Gly-Gly-Gly-Gly-Ser)n (SEQ ID NO:12). By adjusting the copy number “n”, the length of this GS linker can be adjusted to achieve appropriate separation of the functional domains and/or to maintain necessary inter-domain interactions. Besides the GS linkers, many other flexible linkers have been designed for recombinant protein expression.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid which encodes a polypeptide is implicit in a described sequence with respect to the expression product, but not with respect to actual probe sequences.

A “conservative” substitution as used herein refers to a substitution of an amino acid such that charge, hydrophobicity, and/or size of the side group chain is maintained. Illustrative sets of amino acids that may be substituted for one another include (i) positively-charged amino acids Lys, Arg and His; (ii) negatively charged amino acids Glu and Asp; (iii) aromatic amino acids Phe, Tyr and Trp; (iv) nitrogen ring amino acids His and Trp; (v) large aliphatic nonpolar amino acids Val, Leu and Ile; (vi) slightly polar amino acids Met and Cys; (vii) small-side chain amino acids Ser, Thr, Asp, Asn, Gly, Ala, Glu, Gln and Pro; (viii) aliphatic amino acids Val, Leu, Ile, Met and Cys; and (ix) small hydroxyl amino acids Ser and Thr. Reference to the charge of an amino acid in this paragraph refers to the charge at physiological pH.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, and combinations thereof. The terms also include, but is not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. Nucleic acid molecules, e.g. oligonucleotide probes or priomers, may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. A “vector” as used here refers to a recombinant construct in which a nucleic acid sequence of interest is inserted into the vector. Certain vectors are capable of directing the expression of nucleic acids to which they are operatively linked. Such vectors are referred to herein as “expression vectors”.

The terms “identical” or “percent identity,” in the context of two or more nucleic acids, or two or more polypeptides, refer to two or more sequences or subsequences that are the same or have a specified percentage of nucleotides, or amino acids, that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” This definition also refers to, or may be applied to, the compliment of a nucleotide test sequence. The definition also includes sequences that have deletions and/or additions, as well as those that have substitutions. As described below, the algorithms can account for gaps and the like. Typically, identity exists over a region comprising an antibody epitope, or a sequence that is at least about 25 amino acids or nucleotides in length, or over a region that is 50-100 amino acids or nucleotides in length, or over the entire length of the reference sequence.

The terms “corresponding to,” “determined with reference to,” or “numbered with reference to” when used in the context of the identification of a given amino acid residue in a polypeptide sequence, refers to the position of the residue of a specified reference sequence when the given amino acid sequence is maximally aligned and compared to the reference sequence. Thus, for example, an amino acid residue in a variable domain polypeptide “corresponds to” an amino acid in the variable domain polypeptide of SEQ ID NO:1 when the residue aligns with the amino acid in SEQ ID NO:1 when optimally aligned to SEQ ID NO:1. The polypeptide that is aligned to the reference sequence need not be the same length as the reference sequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “isolated,” when applied to a nucleic acid or protein, denotes that the nucleic acid or protein is essentially free of other cellular components with which it is associated in the natural state. It is preferably in a homogeneous state. It can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The terms “therapy,” “treatment,” and “amelioration” refer to any reduction in the severity of symptoms. For example, in the case of treating cancer, e.g., AML, treatment can refer to reducing the number of cancer cells or growth rate or cell death of non-cancer cells, etc. As used herein, the terms “treat” and “prevent” are not intended to be absolute terms. Treatment and prevention can refer to any delay in onset, amelioration of symptoms, improvement in patient survival, increase in survival time or rate, etc. Treatment and prevention can be complete (no detectable symptoms remaining) or partial, such that symptoms are less frequent of severe than in a patient without the treatment described herein. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” “therapeutically effective amount,” etc. refer to that amount of the therapeutic agent sufficient to ameliorate a disorder, as described above. For example, for the given parameter, a therapeutically effective amount will show an increase or decrease of therapeutic effect at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “pharmaceutically acceptable” is used synonymously with physiologically acceptable and pharmacologically acceptable. A pharmaceutical composition will generally comprise agents for buffering and preservation in storage, and can include buffers and carriers for appropriate delivery, depending on the route of administration.

The terms “dose” and “dosage” are used interchangeably herein. A dose refers to the amount of active ingredient given to an individual at each administration. For the present invention, the dose can refer to the concentration of the antibody or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable moiety (if present). One of skill in the art will recognize that the dose can be modified depending on the above factors or based on therapeutic progress. The term “dosage form” refers to the particular format of the pharmaceutical, and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc.

“Cancer”, “tumor,” “transformed” and like terms include precancerous, neoplastic, transformed, and cancerous cells, and can refer to a solid tumor, or a non-solid cancer. Cancer includes both benign and malignant neoplasms (abnormal growth). The term “cancer” can thus refer to carcinomas, sarcomas, adenocarcinomas, lymphomas, leukemias, solid and lymphoid cancers, etc. Examples of different types of cancer include, but are not limited to, lung cancer (e.g., non-small cell lung cancer or NSCLC), ovarian cancer, prostate cancer, colorectal cancer, liver cancer (i.e., hepatocarcinoma), renal cancer (i.e., renal cell carcinoma), bladder cancer, breast cancer, thyroid cancer, pleural cancer, pancreatic cancer, uterine cancer, cervical cancer, testicular cancer, anal cancer, pancreatic cancer, bile duct cancer, gastrointestinal carcinoid tumors, esophageal cancer, gall bladder cancer, appendix cancer, small intestine cancer, stomach (gastric) cancer, cancer of the central nervous system, skin cancer, choriocarcinoma; head and neck cancer, blood cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia (AML), chronic myeloid leukemia (CML), and multiple myeloma. In some embodiments, the antibody compositions and methods described herein can be used for treating cancer.

The term “co-administer” refers to the simultaneous presence of two active agents in the blood of an individual. Active agents that are co-administered can be concurrently or sequentially delivered.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215):215ra172; Glienke et al. Front Pharmacol (2015) 6:21; Kakarla & Gottschalk 52 Cancer J (2014) 20(2):151-5; Riddell et al. Cancer J (2014) 20(2):141-4; Pegram et al. Cancer J (2014) 20(2):127-33; Cheadle et al. Immunol Rev (2014) 257(1):91-106; Barrett et al. Annu Rev Med (2014) 65:333-47; Sadelain et al. Cancer Discov (2013) 3(4):388-98; Cartellieri et al., J Biomed Biotechnol (2010) 956304; the disclosures of which are incorporated herein by reference in their entirety.

As used herein, the term “immune cells” generally includes white blood cells (leukocytes) which are derived from hematopoietic stem cells (HSC) produced in the bone marrow “Immune cells” includes, e.g., lymphocytes (T cells, B cells, natural killer (NK) cells) and myeloid-derived cells (neutrophil, eosinophil, basophil, monocyte, macrophage, dendritic cells).

“T cell” includes all types of immune cells expressing CD3 including T-helper cells (CD4+ cells), cytotoxic T-cells (CD8+ cells), T-regulatory cells (Treg) and gamma-delta T cells.

A “cytotoxic cell” includes CD8+ T cells, natural-killer (NK) cells, and neutrophils, which cells are capable of mediating cytotoxicity responses.

Anti-Itgβ2 Antibodies that Bind Active Conformation

Provided herein are anti-Itgβ2 antibodies that bind the active conformation and can be used for diagnostic and therapeutic purposes.

In some embodiments, an anti-Itgβ2 antibody of the present disclosure has a K_{D} less than about 10 nM.

In some embodiments, an anti-Itgβ2 binding domain of the present disclosure has at least one, at least two, or three CDRs of a variable domain sequence of SEQ ID NO:2 or SEQ ID NO:3. In some embodiments, an anti-Itgβ2 binding domain of the present disclosure comprises an HCDR3 of SEQ ID NO:2 and an LCDR3 of SEQ ID NO:3. In some embodiments, an anti-Itgβ2 binding domain comprises an HCDR1, HCDR2, and HCDR3 of SEQ ID NO:2 and and LCDR1, LCDR2, and LCDR3 of SEQ ID NO:3.

In some embodiments, an anti-Itgβ2 binding domain comprises an HCDR1, HCDR2, and HCDR3 of SEQ ID NO:2 in which one of the CDRs comprises a substitution relative to the corresponding CDR set forth in SEQ ID NO:2. In some embodiments, an anti-Itgβ2 binding domain comprises an HCDR1, HCDR2, and HCDR3 of SEQ ID NO:2 in which two of the CDRs comprise a substitution relative to the the corresponding CDRs set forth in SEQ ID NO:2, and LCDR1, LCDR2, and LCDR3 of SEQ ID NO:3. In some embodiments, an anti-Itgβ2 binding domain comprises an HCDR1, HCDR2, and HCDR3 of SEQ ID NO:2 in which all three of the CDRs comprise a substitution relative to the corresponding CDR sequences set forth in SEQ ID NO:2.

In some embodiments, an anti-Itgβ2 binding domain comprises an LCDR1, LCDR2, and LCDR3 of SEQ ID NO:3 in which one of the CDRs comprises a substitution relative to the corresponding CDR set forth in SEQ ID NO:3. In some embodiments, an anti-Itgβ2 binding domain comprises an LCDR1, LCDR2, and LCDR3 of CDR3 in which two of the CDRs comprise a substitution relative to the the corresponding CDRs set forth in SEQ ID NO:3, and LCDR1, LCDR2, and LCDR3 of SEQ ID NO:3. In some embodiments, an anti-Itgβ2 binding domain comprises an LCDR1, LCDR2, and LCDR3 of SEQ ID NO:3 in which all three of the CDRs comprise a substitution relative to the corresponding CDR sequences set forth in SEQ ID NO:3.

In some embodiments, an anti-Itgβ2 binding domain of the present disclosure comprises a variable region having at least 70%, 75%, 85%, 90%, 91%, 92%, 93%, 94%, 95%, 96%, 97%, 98%, 99%, or 100% sequence identity to the amino acid sequence of a variable region sequence of SEQ ID NO:2 or SEQ ID NO:3. In some embodiments, the variable domain comprises substitutions, insertions, or deletions in the framework of a variable region as shown in SEQ ID NO:2 or SEQ ID NO:3. In some embodiments, an anti-Itgβ2 binding domain of the present disclosure comprises a heavy chain variable region comprising the HCD1, HCDR2, and HCDR3 sequence of SEQ ID NO:2 and having at least 95% identity to SEQ ID NO:2; and a light chain variable region comprising the LCD1, LCDR2, LCDR3 sequences of SEQ ID NO:3 and having at least 95% identity to SEQ ID NO:3.

### Antibody Formats

As previously explained, an anti-Itgβ2 antibody of the present disclosure may be incorporated into a bivalent antibody or a multivalent antibody that binds to the same, or a different, antigen. In some embodiments, an anti-Itgβ2 antibody of the present disclosure may be incorporated into a bispecific antibody or multispecific antibody that binds to the antigen at different epitopes, or that binds to different antigens. In some embodiments, such an antibody may comprise an Fc region. In some embodiments, an anti-Itgβ2 antibody of the present disclosure may be present as an antigen binding domain of a larger molecule, e.g., present as an antigen binding domain of a chimeric antigen receptor or synthetic Notch receptor, as further detailed below.

### CAR Constructs Comprising an Anti-Itgβ2 Antibody of the Present Disclosure Antibody

Chimeric antigen receptors (CARs) are recombinant receptor constructs comprising an extracellular antigen-binding domain (e.g., a antibody) joined to a transmembrane domain, and further linked to an intracellular signaling domain (e.g., an intracellular T cell signaling domain of a T cell receptor) that transduces a signal to elicit a function. In certain embodiments, immune cells (e.g., T cells or natural killer (NK) cells) are genetically modified to express CARs that comprise an anti-Itgβ2 antibody of the present disclosure have the functionality of effector cells (e.g., cytotoxic and/or memory functions of T cells or NK cells).

In a standard CAR, the components include an extracellular targeting domain, a transmembrane domain and intracellular signaling/activation domain, which are typically linearly constructed as a single fusion protein. In the present invention, the extracellular region comprises an anti-Itgβ2 antibody as described herein. The “transmembrane domain” is the portion of the CAR that links the extracellular binding portion and intracellular signaling domain and anchors the CAR to the plasma membrane of the host cell that is modified to express the CAR, e.g., the plasma membrane of an immune effector cell. The intracellular region may contain a signaling domain of TCR complex, and/or one or more costimulatory signaling domains, such as those from CD28, 4-1BB (CD137) and OX-40 (CD134). For example, a “first-generation CAR” generally has a CD3-zeta signaling domain. Additional costimulatory intracellular domains may also be introduced (e.g., second and third generation CARS) and further domains including homing and suicide domains may be included in CAR constructs. CAR components are further described below.

### Extracellular Domain (Antibody Domain)

A chimeric antigen receptor of the present disclosure comprises an extracellular antigen-binding domain that comprises an anti-Itgβ2 binding domain, which binds active confirmation Itgβ2, having heavy and light chain CDRs of SEQ ID NO:2 and SEQ ID NO:3, respectively. In some embodiments, the anti-Itgβ2 binding domain comprises a V_{H} region amino acid SEQ ID NO:2 and a V_{L} region amino acid sequence SEQ ID NO:3. In some embodiments, anti-Itgβ2 binding domain comprises an scFv region as set forth in any one of SEQ ID NOS: 4-11. In some embodiments, the anti-Itgβ2 binding domain comprises an scFv region as set forth in SEQ ID NO:10 or SEQ ID NO:11; or an scFv region that comprises the CDRs set forth in SEQ ID NO:10 or SEQ ID NO:11 and has at least 95% identity to SEQ ID NO:10 or SEQ ID NO:11. In some embodiments, a CAR construct encoding a CAR may also comprise a sequence that encodes a signal peptide to target the extracellular domain to the cell surface.

### Hinge Domain

In some embodiments, the CAR may one or more hinge domains that link the antigen binding domain comprising an anti-Itgβ2 antibody of the present disclosure and the transmembrane domain for positioning the antigen binding domain. Such a hinge domain may be derived either from a natural, synthetic, semi-synthetic, or recombinant source. The hinge domain can include the amino acid sequence of a naturally occurring immunoglobulin hinge region, e.g., a naturally occurring human immunglobulin hinge region, or an altered immunoglobulin hinge region. Illustrative hinge domains suitable for use in the CARs described herein include the hinge region derived from the extracellular regions of type 1 membrane proteins such as CD8 alpha, CD4, CD28, PD-1, CD152, and CD7, which may be wild-type hinge regions from these molecules or may be altered.

### Transmembrane Domain

Any transmembrane suitable for use in a CAR construct may be employed. Such transmembrane domains, include, but are not limited to, all or part of the transmembrane domain of the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGAl, VLAI, CD49a, ITGA4, IA4, CD49D, ITGA6, VLA-6, CD49f, ITGAD, CD11d, ITGAE, CD103, ITGAL, CD1 1a, LFA-1, ITGAM, CD1 1b, ITGAX, CD1 1c, ITGB 1, CD29, ITGB2, CD 18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CD100, (SEMA4D), SLAMF6 (NTB-A, Ly108), SLAM (SLAMF1, CD150, IPO-3), BLAME, (SLAMF8), SELPLG (CD162), LTBR, PAG/Cbp, NKG2D, or NKG2C.

A transmembrane domain incorporated into a CAR construct may be derived either from a natural, synthetic, semi-synthetic, or recombinant source.

### Intracellullar Signaling Domain

A CAR construct of the present disclosure includes one or more intracellular signaling domains, also referred to herein as co-stimulatory domains, or cytoplasmic domains that activate or otherwise modulate an immune cell, (e.g., a T lymphocyte or NK cell). The intracellular signaling domain is generally responsible for activation of at least one of the normal effector functions of the immune cell in which the CAR has been introduced. In one embodiment, a co-stimulatory domain is used that increases CAR immune T cell cytokine production. In another embodiment, a co-stimulatory domain is used that facilitates immune cell (e.g., T cell) replication. In still another embodiment, a co-stimulatory domain is used that prevents CAR immune cell (e.g., T cell) exhaustion. In another embodiment, a co-stimulatory domain is used that increases immune cell (e.g., T cell) antitumor activity. In still a further embodiment, a co-stimulatory domain is used that enhances survival of CAR immune cells (e.g., T cells) (e.g., post-infusion into patients).

Examples of intracellular signaling domains for use in a CAR include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM-containing primary intracellular signaling domains include those of CD3 zeta, common FcR gamma, Fc gamma Rlla, FcR beta (Fc Epsilon Rib), CD3 gamma, CD3 delta, CD3 epsilon, CD79a, CD79b, DAP10, and DAP12. In one embodiment, a CAR comprises an intracellular signaling domain, e.g., a primary signaling domain of CD3-zeta.

An intracellular signaling domain of a CAR can comprise a primary intracellular signaling domain only, or may comprise additional desired intracellular signaling domain(s) useful in the context of a CAR of the invention. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that binds to CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include CDS, ICAM-1, GITR, BAFFR, HVEM (LIGHTR), SLAMF7, NKp80 (KLRF1), NKp44, NKp30, NKp46, CD 160, CD 19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD1 1b, ITGAX, CD1 1c, ITGB 1, CD29, ITGB2, CD18, LFA-1, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), CD160 (BY55), PSGL1, CD100 (SEMA4D), CD69, SLAMF6 (NTB-A, Lyl08), SLAM, (SLAMF1, CD150, IPO-3), BLAME (SLAMF8), SELPLG (CD162), LTBR, LAT, GADS, SLP-76, PAG/Cbp, and CD19a.

In some embodiments, a CAR may be designed as an inducible CAR, or may otherwise comprise a mechanism for reversibly expressing the CAR, or controlling CAR activity to largely restrict it to a desired environment. Thus, for example, in some embodiments, the CAR-expressing cell uses a split CAR. The split CAR approach is described in more detail in publications WO2014/055442 and WO2014/055657. Briefly, a split CAR system comprises a cell expressing a first CAR having a first antigen binding domain and a costimulatory domain (e.g., 41BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

In some embodiments, a host cell, e.g., a T cell, can be engineered such that a synthetic Notch receptor comprising an extracellular domain that targets one antigen induces the expression of a CAR that targets a second antigen. Such systems are described, e.g., in U.S. Patent Application Publication No. 20190134093; see also, synNotch polypeptides as described in US20160264665, each incorporated herein by reference. In some embodiments, a synNotch receptor comprises an anti-Itgβ2 antibody as described herein. In some embodiments, an anti-Itgβ2 antibody is incorporated into a CAR, the expression of which is activated by an antigen triggering a synNotch receptor expressed by the host cell.

In some embodiments, a cell expressing a CAR comprising an anti-Itgβ2 antibody as described herein also expresses a second CAR, e.g., a second CAR that includes a different antigen binding domain, e.g., that binds to the same target or a different target that is expressed on cancer cells, e.g., AML cells.

### Activation and Expansion of Immune Effector Cells (e.g., T Cells)

The invention is not limited by the type of immune cells genetically modified to express a CAR, or synthetic Notch receptor. Illustrative immune cells include, but are not limited to, T cells, e.g., alpha/beta T cells and gamma/delta T cells, B cells, natural killer (NK) cells, natural killer T (NKT) cells, mast cells, macrophages, and myeloid-derived phagocytes. T cells that can be modified to express CARs include memory T cells, CD4+, and CD8+ T cells. In some embodiments, the immune cells, e.g., T cells, are autologous cells from the patient to undergo immunotherapy. In some embodiments, the immune cells are allogeneic.

Immune effector cells such as T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 2006/0121005. Examples of immune effector cells include T cells, e.g., alpha/beta T cells and gamma/delta T cells, B cells, natural killer (NK) cells, natural killer T (NKT) cells, mast cells, and myeloid-derived phagocytes.

Methods of making CAR-expressing cells are described, e.g., in US2016/0185861 and US2019/0000880.

### Nucleic Acids and Vectors Encoding CARs

Any method may be used to genetically modify an effector cells, such as a T-cell or NK cell to express a CAR comprising an anti-Itgβ2 antibody of the present disclosure. Non-limiting examples of methods of genetically engineering immune cells include, but are not limited to, retrovirus- or lentivirus-mediated transduction. Other viral delivery systems include adenovirus, adeno-associated virus, herpes simplex viral vectors, pox viral vectors, alphavirus vectors, poliovirus vectors, and other positive and negative stranded RNA viruses, viroids, and virusoids, or portions thereof. Methods of transduction include direct co-culture of the cells with producer cells, e.g., by the method of Bregni, et al. Blood 80: 1418-1422 (1992), or culturing with viral supernatant alone or concentrated vector stocks with or without appropriate growth factors and polycations, e.g., by the method of Xu, et al. Exp. Hemat. 22:223-230 (1994); and Hughes, et al. J. Clin. Invest. 89: 1817 (1992).

In some embodiments, genetic modification is performed using transposase-based systems for gene integration, CRISPR/Cas-mediated gene integration, TALENs or Zinc-finger nucleases integration techniques. For example, CRISPR/Cas-mediated gene integration may be employed to introduce a CAR or synthetic Notch receptor into immune effectors cells, which may then be selected and expanded for administration to a patient.

### Antibody Conjugates

In a further aspect, an anti-Itgβ2 antibody of the present disclosure may be conjugated or linked, either directly or indirectly, to therapeutic and/or imaging/detectable moieties. For example, in some embodiments, an antibody of the present disclosure, or an antigen binding region comprising a antibody of the present invention, may be conjugated to agents including, but not limited to, a detectable marker, a cytotoxic agent, an imaging agent, a therapeutic agent, or an oligonucleotide. Methods for conjugating or linking an antibody, or antigen binding regions comprising an antibody, to a desired molecule moiety are well known in the art. The moiety may be linked to the antibody covalently or by non-covalent linkages.

In some embodiments, an anti-Itgβ2 antibody of the present disclosure, or an antigen binding domain comprising an anti-Itgβ2 antibody of the present disclosure, is conjugated to cytotoxic moiety or other moiety that inhibits cell proliferation. In some embodiments, the antibody is conjugated to a cytotoxic agent including, but not limited to, e.g., ricin A chain, doxorubicin, daunorubicin, a maytansinoid, taxol, ethidium bromide, mitomycin, etoposide, tenoposide, vincristine, vinblastine, colchicine, dihydroxy anthracin dione, methotrexact, actinomycin, a diphtheria toxin, extotoxin A from Pseudomonas, Pseudomonas exotoxin40, abrin, abrin A chain, modeccin A chain, alpha sarcin, gelonin, mitogellin, restrictocin, cobran venom factor, a ribonuclease, engineered Shiga toxin, phenomycin, enomycin, curicin, crotin, calicheamicin, Saponaria officinalis inhibitor, glucocorticoid, auristatin, auromycin, yttrium, bismuth, combrestatin, duocarmycins, dolastatin, cc1065, or a cisplatin. In some embodiments, the antibody may be linked to an agent such as an enzyme inhibitor, a proliferation inhibitor, a lytic agent, a DNA or RNA synthesis inhibitors, a membrane permeability modifier, a DNA metabolite, a dichloroethylsulfide derivative, a protein production inhibitor, a ribosome inhibitor, or an inducer of apoptosis.

In some embodiments, an anti-Itgβ2 antibody of the present disclosure, or an antigen binding domain comprising an anti-Itgβ2 antibody of the present disclosure, may be linked to a radionuclide, an iron-related compound, a dye, a fluorescent agent, or an imaging agent. In some embodiments, an antibody may be linked to agents, such as, but not limited to, metals; metal chelators; lanthanides; lanthanide chelators; radiometals; radiometal chelators; positron-emitting nuclei; microbubbles (for ultrasound); liposomes; molecules microencapsulated in liposomes or nanosphere; monocrystalline iron oxide nanocompounds; magnetic resonance imaging contrast agents; light absorbing, reflecting and/or scattering agents; colloidal particles; fluorophores, such as near-infrared fluorophores.

### Cancer Vaccines

Ann anti-Itgβ2 antibody of the present disclosure, an antigen binding molecule comprising such an anti-Itgβ2 antibody, or an effector cell, e.g., T-cell, genetically modified to comprise a CAR comprising an anti-Itgβ2 antibody of the present disclosure can be combined with an immunogenic agent, such as cancerous cells, purified tumor antigens (including recombinant proteins, peptides, and carbohydrate molecules), and cells transfected with genes encoding immune stimulating cytokines (He et al. (2004) J. Immunol. 173:4919-28). Non-limiting examples of cancer vaccines that can be used include t cells transfected to express the cytokine GM-CSF, DNA-based vaccines, RNA-based vaccines, and viral transduction-based vaccines. The cancer vaccine may be prophylactic or therapeutic.

In some embodiments, an anti-Itgβ2 antibody of the present disclosure, an antigen binding molecule comprising such an antibody, or an effector cell, e.g., T-cell, genetically modified to comprise a CAR comprising an an anti-Itgβ2 antibody of the present disclosure is co-administered with an immunomodulating agent. Examples of immodulating agents include, but are not limited to, cytokines, growth factors, lymphotoxins, tumor necrosis factor (TNF), hematopoietic factors, interleukins (e.g., interleukin-1 (IL-1), IL-2, IL-3, IL-6, IL-10, IL-12, IL-15, an IL-15/IL-15Rα, e.g., sushi domain, complex, IL-18, and IL-21), colony stimulating factors (e.g., granulocyte-colony stimulating factor (G-CSF) and granulocyte macrophage-colony stimulating factor (GM-CSF), interferons (e.g., interferon-α, -β or -γ), erythropoietin and thrombopoietin, or a combination thereof. In some embodiments, the complex may be co-administered with an adjuvant, such as a Toll-like receptor (TLR) agonist, a C-type lectin receptor (CLR) agonist, a retinoic acid-inducible gene I-like receptor (RLR) agonist, a saponin, a polysaccharide such as chitin, chitosan, β-glucan, an ISCOM, QS-21, or another immunopotentiating agent.

### Treatment of Cancer

An anti-Itgβ2 antibody of the present disclosure, including embodiments in which the antibody is provided as a component of an antigen binding molecule, such as a bivalent or multivalent antibody, or is provided as a component of a CAR molecule, can be used to treat any malignancy that expresses active conformation Itgβ2. In some embodiments, the malignancy is acute myeloid leukemia.

### Administration of Anti-Itgβ22 Antibody

In one aspect, a method of treating a cancer, e.g., AML, using an anti Itgβ2 antibody of the present disclosure or antigen binding molecule, e.g., an antibody, that comprises the Itgβ2 antibody, comprises administering the antibody or antigen binding molecule as a pharmaceutical composition to a patient in a therapeutically effective amount using a dosing regimen suitable for treatment of the cancer, e.g., AML. The composition can be formulated for use in a variety of drug delivery systems. One or more physiologically acceptable excipients or carriers can also be included in the compositions for proper formulation. Suitable formulations for use in the present invention are found, e.g., in Remington: The Science and Practice of Pharmacy, 21st Edition, Philadelphia, PA. Lippincott Williams & Wilkins, 2005.

The antibody (or antibody or antigen binding molecule comprising the antibody) is provided in a solution suitable for administration to the patient, such as a sterile isotonic aqueous solution for injection. The antibody is dissolved or suspended at a suitable concentration in an acceptable carrier. In some embodiments the carrier is aqueous, e.g., water, saline, phosphate buffered saline, and the like. The compositions may contain auxiliary pharmaceutical substances as required to approximate physiological conditions, such as pH adjusting and buffering agents, tonicity adjusting agents, and the like.

The pharmaceutical compositions are administered to a patient in an amount sufficient to cure or at least partially arrest the disease or symptoms of the disease and its complications. An amount adequate to accomplish this is defined as a “therapeutically effective dose.” A therapeutically effective dose is determined by monitoring a patient's response to therapy. Typical benchmarks indicative of a therapeutically effective dose include the amelioration of symptoms of the disease in the patient. Amounts effective for this use will depend upon the severity of the disease and the general state of the patient's health, including other factors such as age, weight, gender, administration route, etc. Single or multiple administrations of the antibody may be administered depending on the dosage and frequency as required and tolerated by the patient. In any event, the methods provide a sufficient quantity of anti-Itgβ2 antibody or antigen binding molecule that comprises the anti-Itgβ2 antibody to effectively treat the patient.

The antibody can be administered by any suitable means, including, for example, parenteral, intrapulmonary, and intranasal administration. Parenteral infusions include intramuscular, intravenous, intraarterial, intraperitoneal, or subcutaneous administration. In some embodiments, the antibody may be administered by insufflation. In an illustrative embodiment, the antibody may be stored at 10 mg/ml in sterile isotonic aqueous saline solution for injection at 4° C. and is diluted in either 100 ml or 200 ml 0.9% sodium chloride for injection prior to administration to the patient. In some embodiments, the antibody is administered by intravenous infusion over the course of 1 hour at a dose of between 0.01 and 25 mg/kg. In other embodiments, the antibody is administered by intravenous infusion over a period of between 15 minutes and 2 hours. In still other embodiments, the administration procedure is via sub-cutaneous bolus injection.

The dose of antibody is chosen in order to provide effective therapy for the patient and is in the range of less than 0.01 mg/kg body weight to about 25 mg/kg body weight or in the range 1 mg-2 g per patient. Preferably the dose is in the range 0.1-10 mg/kg or approximately 50 mg-1000 mg/patient. The dose may be repeated at an appropriate frequency which may be in the range once per day to once every three months, or every six months, depending on the pharmacokinetics of the antibody (e.g., half-life of the antibody in the circulation) and the pharmacodynamic response (e.g., the duration of the therapeutic effect of the antibody). In some embodiments, the in vivo half-life of between about 7 and about 25 days and antibody dosing is repeated between once per week and once every 3 months or once every 6 months. In other embodiments, the antibody is administered approximately once per month.

### Administration of Immune Effector Cells Comprising an Anti-Itgβ22 Antibody

In some embodiments, pharmaceutical compositions of the present invention comprise a CAR-expressing immune effector cells e.g., a plurality of CAR-expressing immune effector cells that are genetically modified to express a CAR comprising an Itgβ2 antibody as described herein. Such cells may be formulated with one or more pharmaceutically or physiologically acceptable carriers, diluents or excipients, e.g., buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In some embodiments, immune effector cells genetically modified to express a CAR comprising an anti Itgβ2 antibody of the present disclosure are formulated for intravenous administration.

Pharmaceutical compositions comprising the CAR-modified immune effector cells may be administered in a manner appropriate for the cancer, e.g., AML, to be treated. The quantity and frequency of administration will be determined by such factors as the condition of the patient, and the type and severity of the patient's disease, although appropriate dosages may be determined by clinical trials.

In some embodiments, a pharmaceutical composition comprising CAR-modified immune effector cells, e.g., T cells or NK cells as described herein, are administered at a dosage of 10⁴ to 10⁹ cells/kg body weight, in some instances 10⁵ to 10⁶ cells/kg body weight, including all integer values within those ranges. In some embodiments, the cells, e.g., T cells or NK cells modified as described herein, may be administered at 3×10⁴, 1×10⁶, 3×10⁶, or 1×10⁷ cells/kg body weight. The cell compositions may also be administered multiple times at these dosages. Administration can be performed using infusion techniques that are commonly known in immunotherapy (see, e.g., Rosenberg et al., New Eng. J of Med. 319: 1676, 1988). In some embodiments, the genetically modified immune effector cells are administered intravenously. In such cells are administered to a patient by intradermal or subcutaneous injection. The CAR-expressing cells may also be injected directly into a particular site, such as a lymph node.

In some embodiments, a, subject may undergo leukapheresis, wherein leukocytes are collected, enriched, or depleted ex vivo to select and/or isolate the cells of interest, e.g., T or NK cells. These cell isolates, e.g., T cell or NK cell isolates, may be expanded by methods known in the art and treated such that one or more CAR constructs of the invention may be introduced, thereby creating a CAR-expressing cell, e.g., CAR-T cell or CAR-expressing NK cell, of the invention. Subjects in need thereof may subsequently undergo standard treatment with high dose chemotherapy followed by peripheral blood stem cell transplantation. In certain aspects, following or concurrent with the transplant, subjects receive an infusion of the expanded CAR-expressing cells of the present invention. In an additional aspect, expanded cells are administered before or following surgery.

In embodiments, lymphodepletion, e.g., using melphalan, cytoxan, cyclophosphamide, or fludarabind, is performed on a subject, e.g., prior to administering a population of immune effectors cells that express a CAR comprising an anti-Itgβ2 antibody of the present disclosure.

In one embodiment, a CAR is introduced into cells, e.g., T cells or NK cells, e.g., using in vitro transcription, and the subject (e.g., human) receives an initial administration of CAR-expressing cells, e.g., CAR T cells or CAR-expressing NK cells of the invention, and one or more subsequent administrations of the CAR-expressing cells, e.g., CAR T cells or CAR-expressing NK cells of the invention, wherein the one or more subsequent administrations are administered less than 15 days, e.g., 14, 13, 12, 11, 10, 9, 8, 7, 6, 5, 4, 3, or 2 days after the previous administration. In one embodiment, more than one administration of the CAR-expressing cells, e.g., CAR T cells or CAR-expressing NK cells of the invention are administered to the subject (e.g., human) per week, e.g., 2, 3, or 4 administrations of the CAR-expressing cells, e.g., CAR T cells or CAR-expressing NK cells of the invention are administered per week. In one embodiment, the subject (e.g., human subject) receives more than one administration of the CAR-expressing cells, e.g., CAR T cells per week or CAR-expressing NK cells (e.g., 2, 3 or 4 administrations per week) (also referred to herein as a cycle), followed by a week of no CAR-expressing cells, e.g., CAR T cell administrations or CAR-expressing NK cell administrations, and then one or more additional administration of the CAR-expressing cells, e.g., CAR T cells or CAR-expressing NK cells (e.g., more than one administration of the CAR-expressing cells, e.g., CAR T cells or CAR-expressing NK cells, per week) is administered to the subject. In another embodiment, the subject (e.g., human subject) receives more than one cycle of CAR-expressing cells, e.g., CAR T cells or CAR-expressing NK cells, and the time between each cycle is less than 10, 9, 8, 7, 6, 5, 4, or 3 days. In one embodiment, the CAR-expressing cells, e.g., CAR-T cells or CAR-expressing NK cells, are administered every other day for 3 administrations per week. In one embodiment, the CAR-expressing cells, e.g., CAR T cells or CAR-expressing NK cells of the invention, are administered for at least two, three, four, five, six, seven, eight or more weeks.

In some embodiments, CAR-expressing cells as disclosed herein can be administered or delivered to the subject via a biopolymer scaffold, e.g., a biopolymer implant. Biopolymer scaffolds can support or enhance the delivery, expansion, and/or dispersion of the CAR-expressing cells described herein. A biopolymer scaffold comprises a biocompatible (e.g., does not substantially induce an inflammatory or immune response) and/or a biodegradable polymer that can be naturally occurring or synthetic. Examples of suitable biopolymers include, but are not limited to, agar, agarose, alginate, alginate/calcium phosphate cement (CPC), beta-galactosidase (β-GAL), (1,2,3,4,6-pentaacetyl a-D-galactose), cellulose, chitin, chitosan, collagen, elastin, gelatin, hyaluronic acid collagen, hydroxyapatite, poly(3-hydroxybutyrate-co-3-hydroxy-hexanoate) (PHBHHx), poly(lactide), poly(caprolactone) (PCL), poly(lactide-co-glycolide) (PLG), polyethylene oxide (PEO), poly(lactic-co-glycolic acid) (PLGA), polypropylene oxide (PPO), polyvinyl alcohol) (PVA), silk, soy protein, and soy protein isolate, alone or in combination with any other polymer composition, in any concentration and in any ratio. The biopolymer can be augmented or modified with adhesion- or migration-promoting molecules, e.g., collagen-mimetic peptides that bind to the collagen receptor of lymphocytes, and/or stimulatory molecules to enhance the delivery, expansion, or function, e.g., anti-cancer activity, of the cells to be delivered. The biopolymer scaffold can be an injectable, e.g., a gel or a semi-solid, or a solid composition.

In some embodiments, CAR-expressing cells described herein are seeded onto the biopolymer scaffold prior to delivery to the subject. In embodiments, the biopolymer scaffold further comprises one or more additional therapeutic agents described herein (e.g., another CAR-expressing cell, an antibody, or a small molecule) or agents that enhance the activity of a CAR-expressing cell, e.g., incorporated or conjugated to the biopolymers of the scaffold. In embodiments, the biopolymer scaffold is injected, e.g., intratumorally, or surgically implanted at the tumor or within a proximity of the tumor sufficient to mediate an anti-tumor effect. Additional examples of biopolymer compositions and methods for their delivery are described in Stephan et al., Nature Biotechnology, 2015, 33:97

Administration in Combination with Other Agents

An Itgβ2 antibody of the present disclosure (or antibody or antigen binding molecule comprising the antibody), or immune effector cells genetically modified to express a antibody as described herein may be administered with one or more additional therapeutic agents, e.g., radiation therapy, chemotherapeutic agents and/or immunotherapeutic agents. As used herein, administered “in combination”, means that two (or more) different treatments are delivered to the subject for the treatment of the cancer, e.g., AML, e.g., the two or more treatments are administered after the subject has been diagnosed with the cancer. In some embodiments, there may be overlap in the time frames in which the two therapeutic agents are administered. In other embodiments, one treatment protocol ends before the second begins. In some embodiment, treatment may be more effective because of combined administration.

In some embodiments, the antibody or immune effector cells that express a CAR comprising the antibody, are administered in conjunction with an agent that targets an immune checkpoint antigen. In one aspect, the agent is a biologic therapeutic or a small molecule. In another aspect, the agent is a monoclonal antibody, a humanized antibody, a human antibody, a fusion protein or a combination thereof. In certain embodiments, the agents inhibit, e.g., by blocking ligand binding to receptor, a checkpoint antigen that may be PD1, PDL1, CTLA-4, ICOS, PDL2, IDO1, IDO2, B7-H3, B7-H4, BTLA, HVEM, TIM3, GAL9, GITR, HAVCR2, LAG3, KIR, LAIR1, LIGHT, MARCO, OX-40, SLAM, 2B4, CD2, CD27, CD28, CD30, CD40, CD70, CD80, CD86, CD137 (4-1BB), CD160, CD39, VISTA, TIGIT, a SIGLEC, CGEN-15049, 2B4, CHK 1, CHK2, A2aR, B-7 family ligands or a combination thereof. In some embodiments, the agent targets PD-1, e.g., an antibody that blocks PD-L1 binding to PD-1 or otherwise inhibits PD-1. In some embodiments, agent targets CTLA-4. In some embodiments, the targets LAG3. In some embodiments, the agents targets TIM3. In some embodiments, the agents target ICOS.

In some embodiments, the anti-Itgβ2 antibody or immune effector cells expressing a CAR comprising the antibody can be administered in conjunction with an additional therapeutic antibody that targets an antigen on a cancer, such as AML.

In some embodiments, the anti-Itgβ2 antibody or immune effector cells comprising the antibody are administered with a chemotherapeutic agent. Examples of cancer chemotherapeutic agents include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; antimetabolites such as methotrexate and 5-fluorouracil; folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside; cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; docetaxel, platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylomithine (DMFO); retinoic acid derivatives such as bexarotene, alitretinoin; denileukin diftitox; esperamicins; capecitabine; and pharmaceutically acceptable salts, acids or derivatives of any of the above.

The following technical section represents certain aspects of the present disclosure. It is understood that the examples and embodiments described herein are for illustrative purposes only and that various modifications or changes in light thereof will be suggested to persons skilled in the art and are to be included within the spirit and purview of this application and scope of the appended claims.

### Techniques/Technical Disclosure

In our pursuit of immunotherapy development for AML, we tried to repurpose two of the existing activated Itgβ2 specific antibodies namely M24 (Hogg et al., Cellular Immunol 1985) and AL-57 (Shimaoka et al., Proc. Nat. Acad Sci 2006) in CAR-T format, but we found that neither antibody was functional in CAR-T format. Here we describe the identification and characterization of antibodies specific for activated Itgβ2.

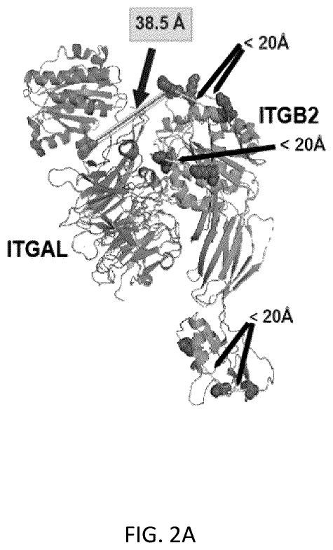

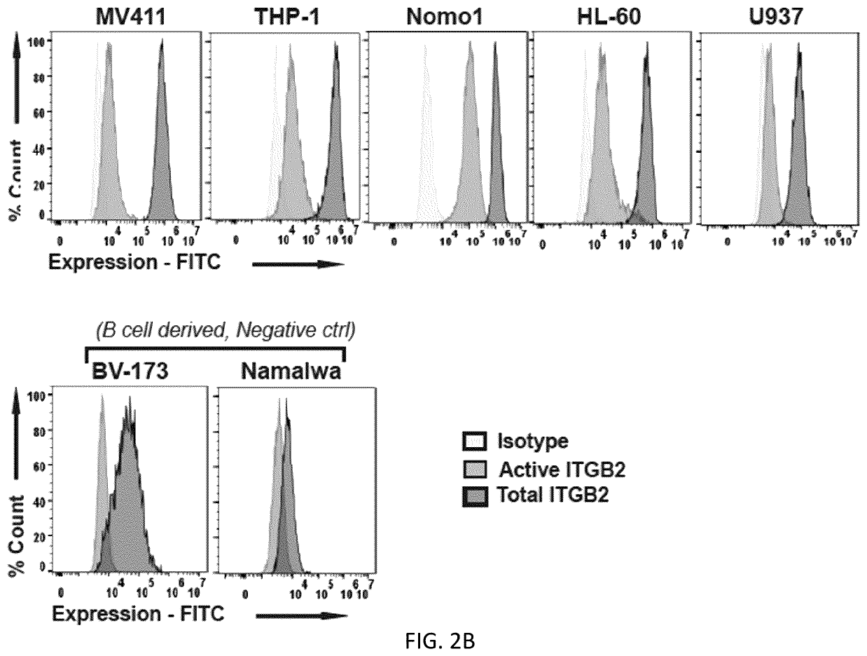

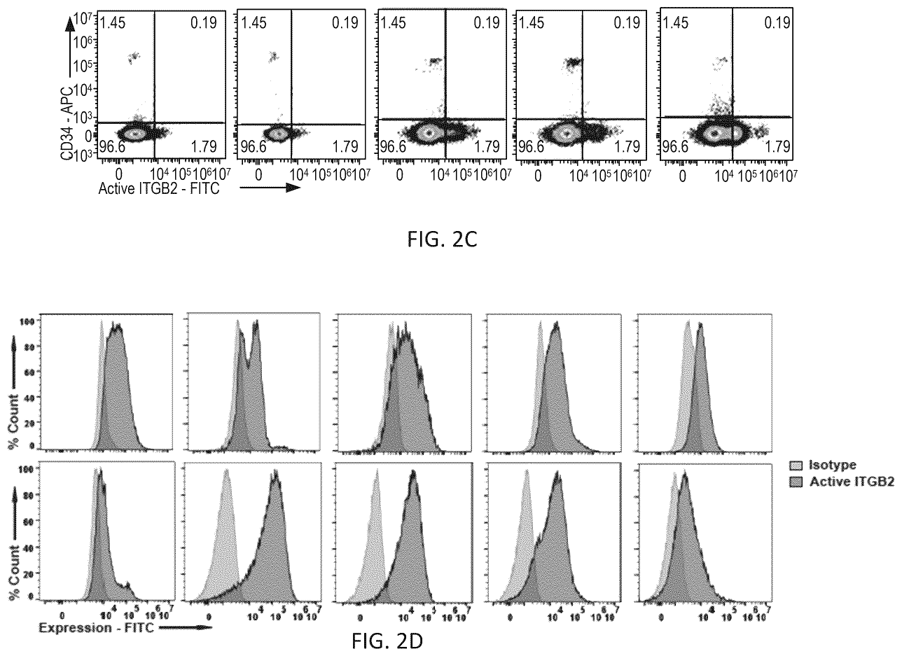

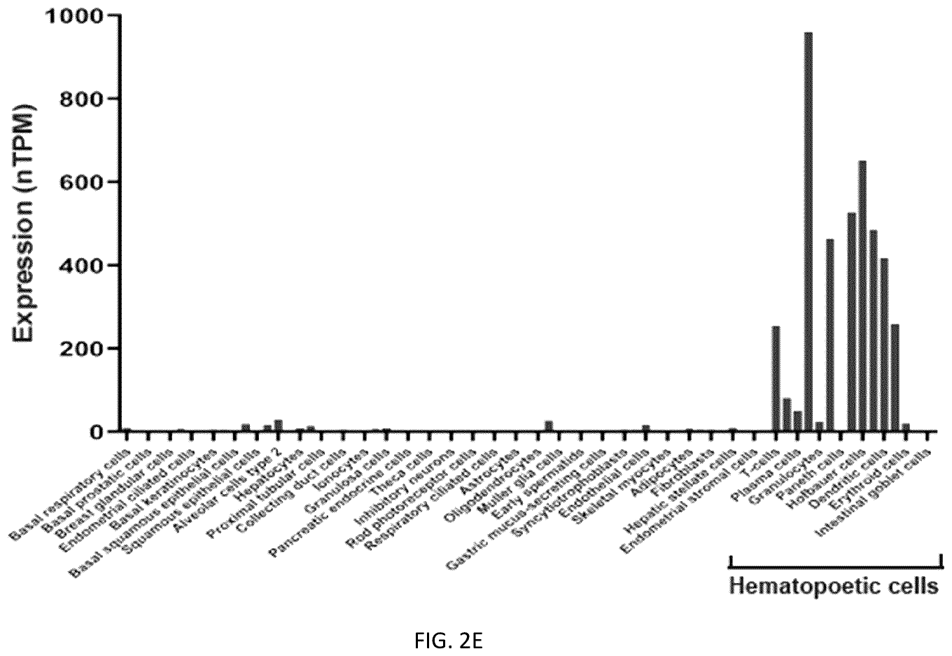

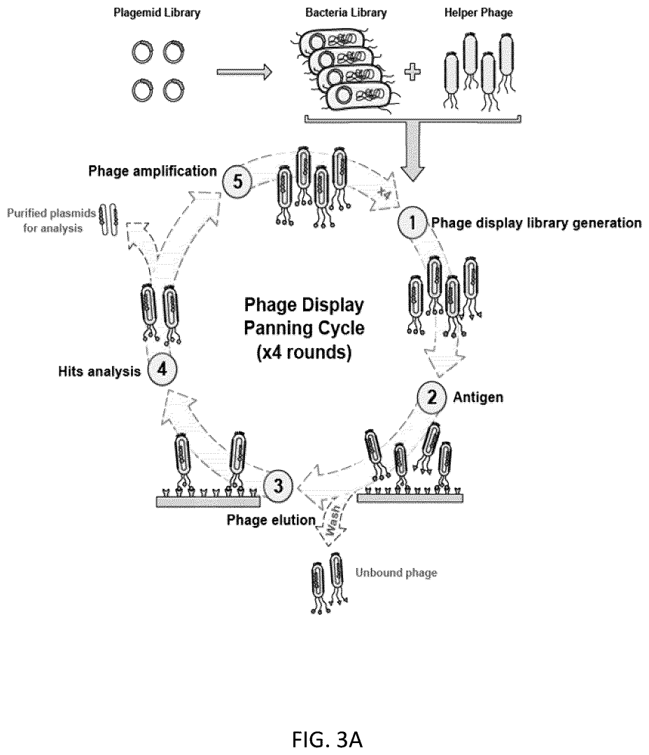

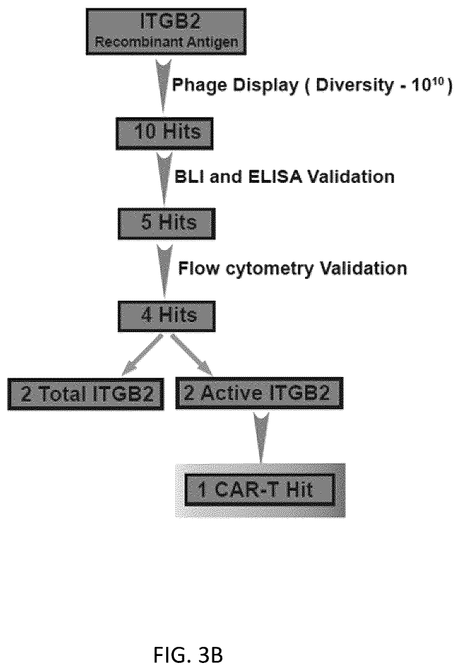

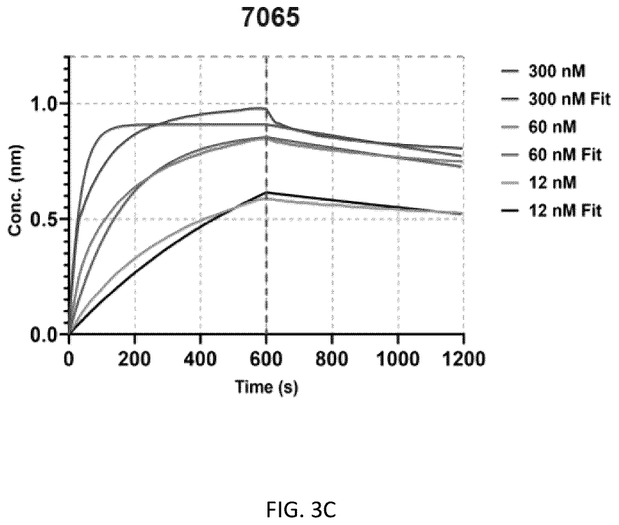

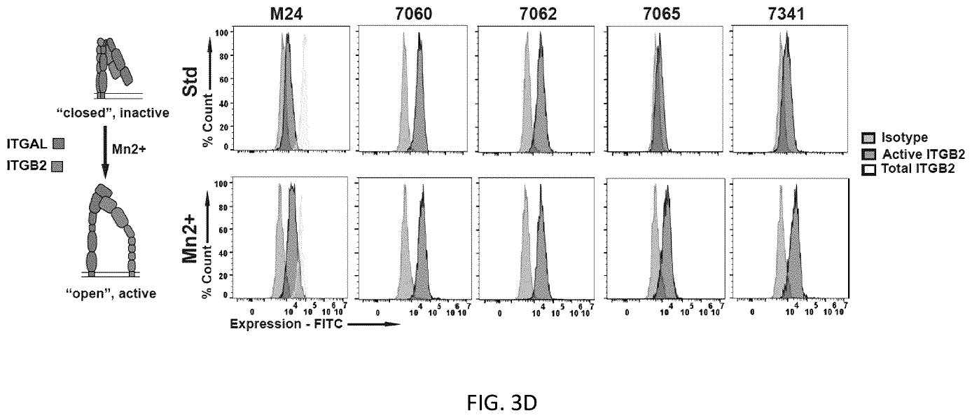

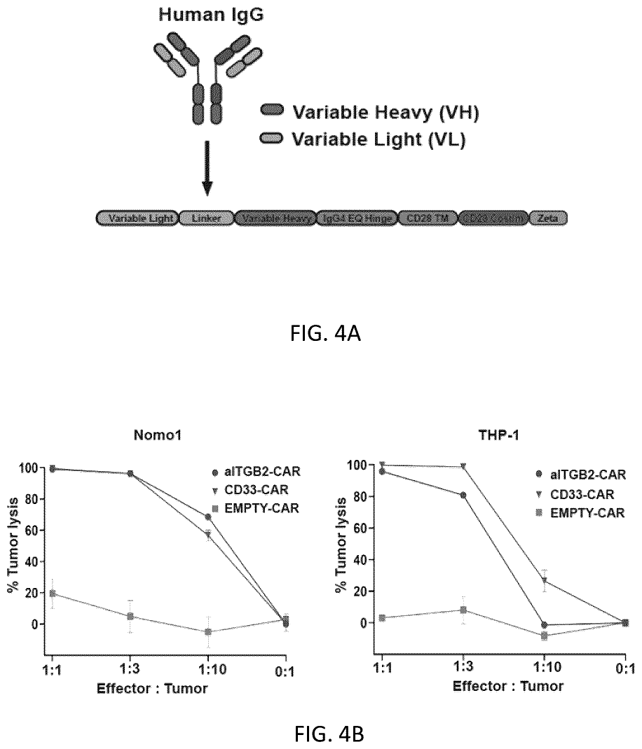

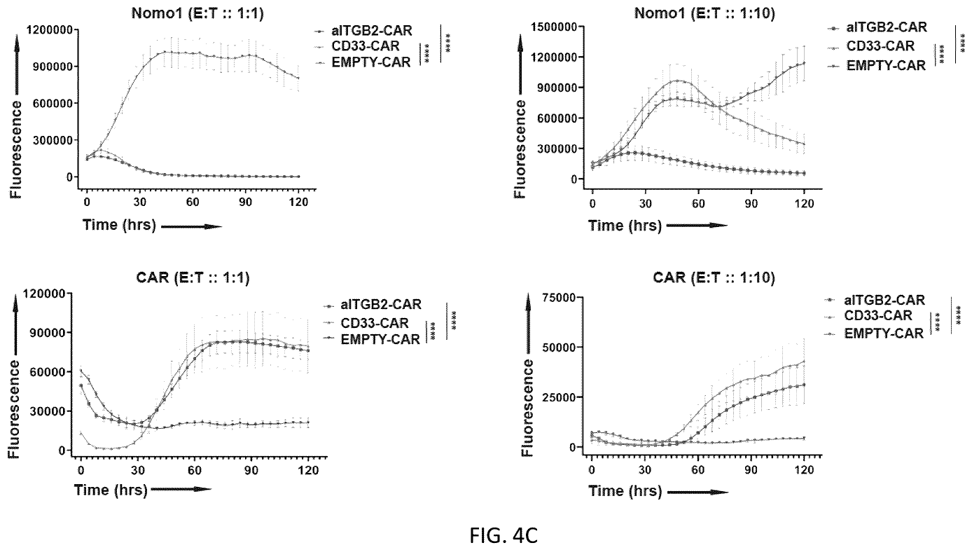

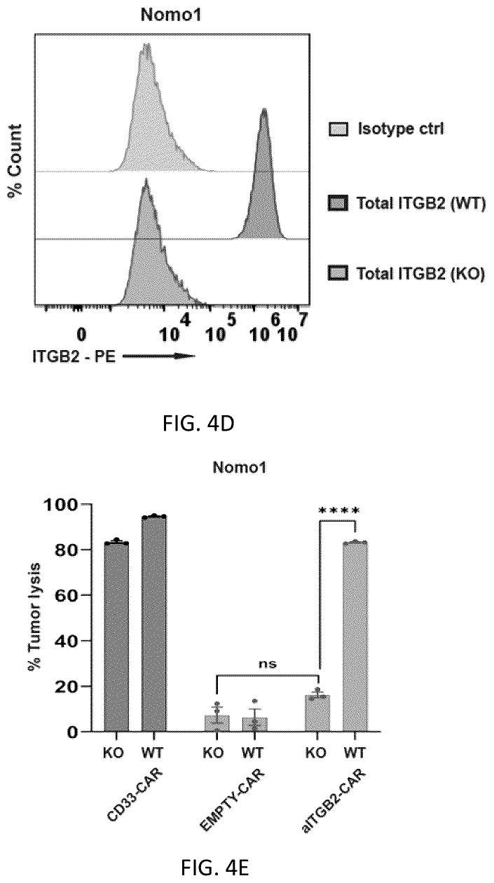

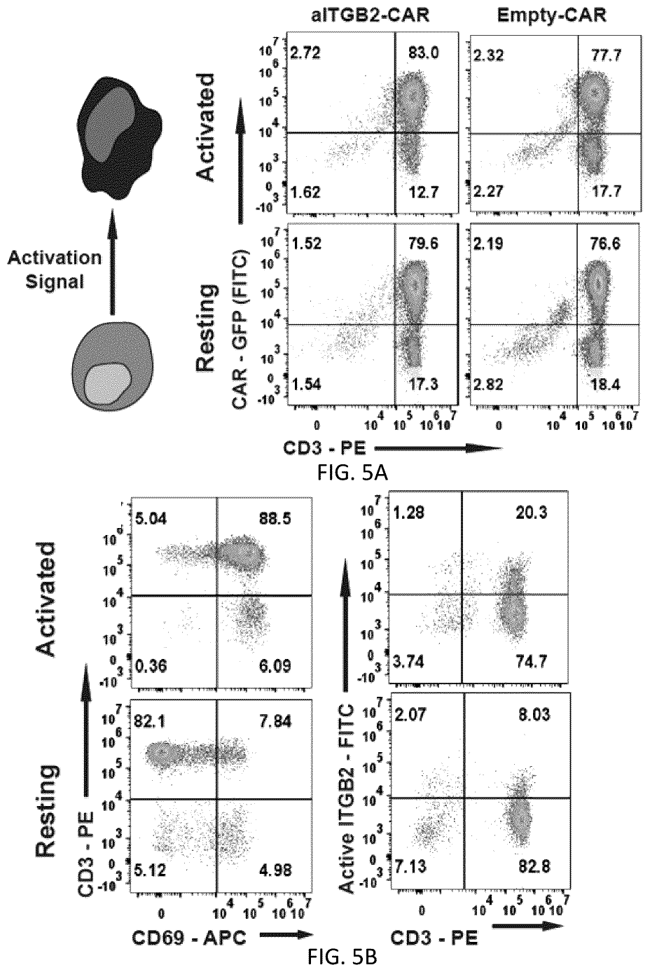

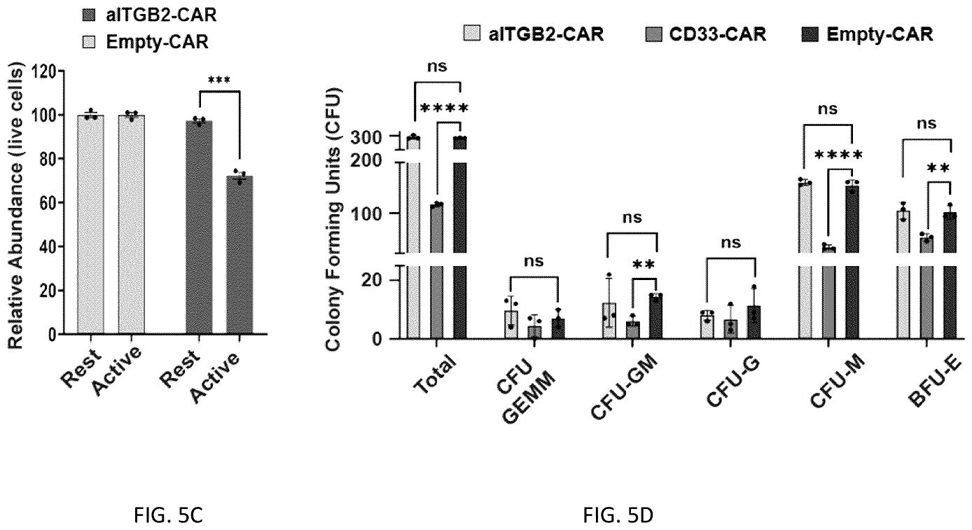

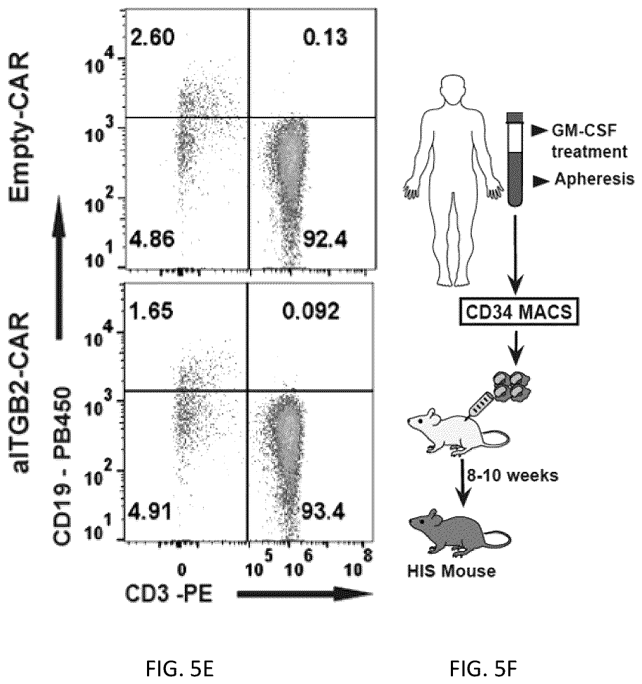

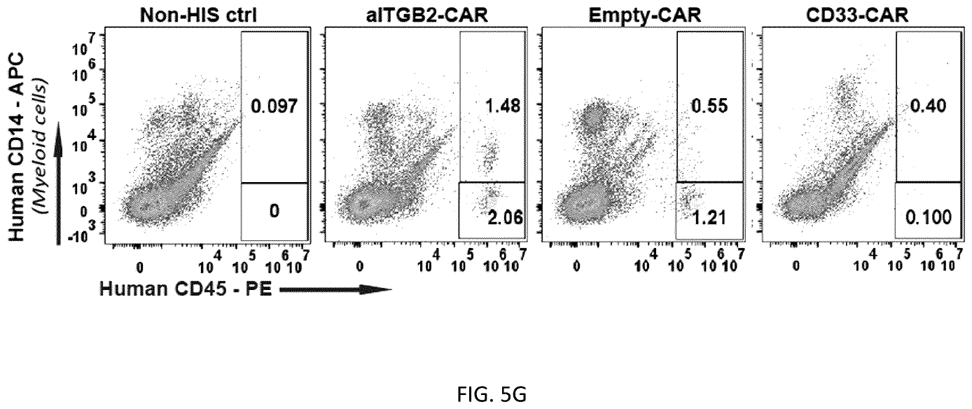

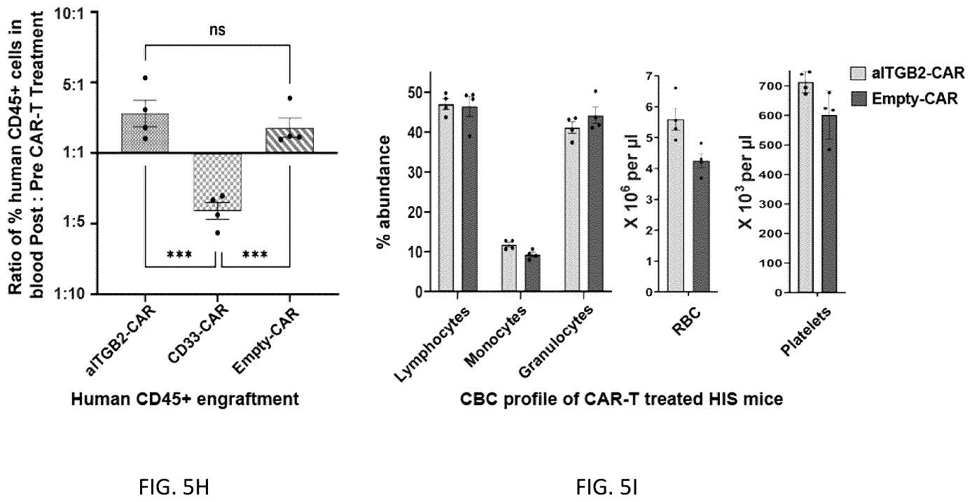

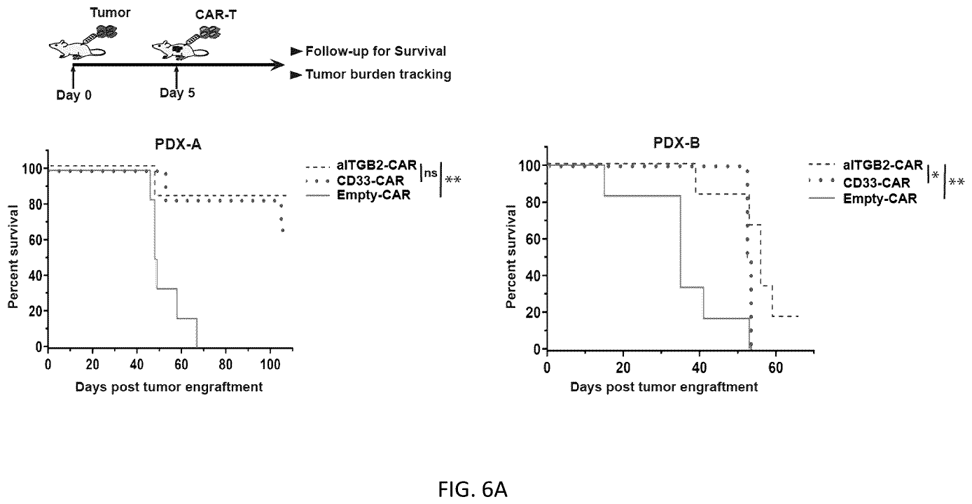

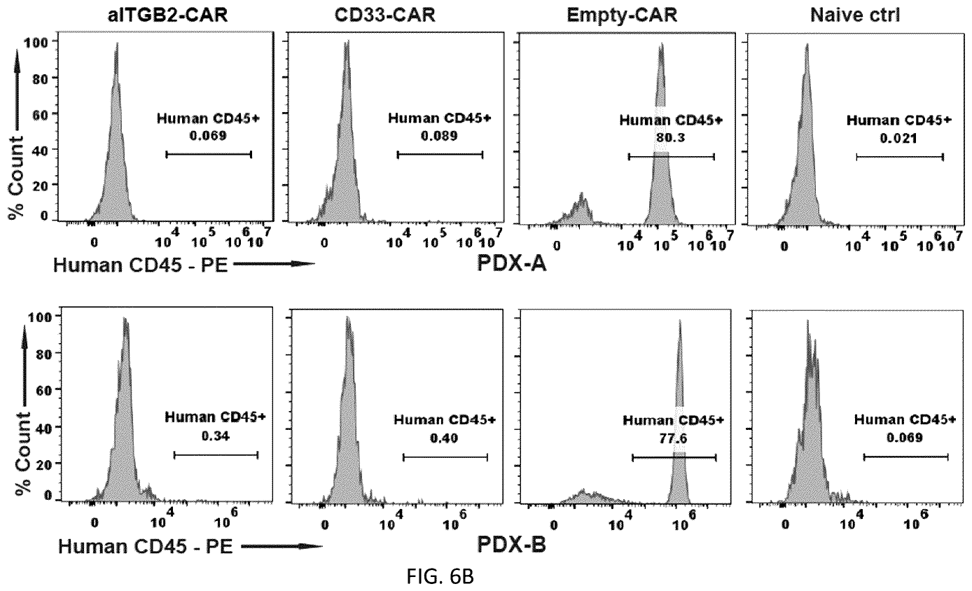

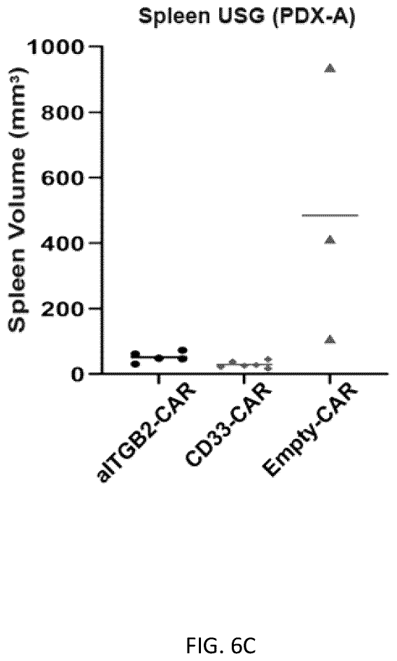

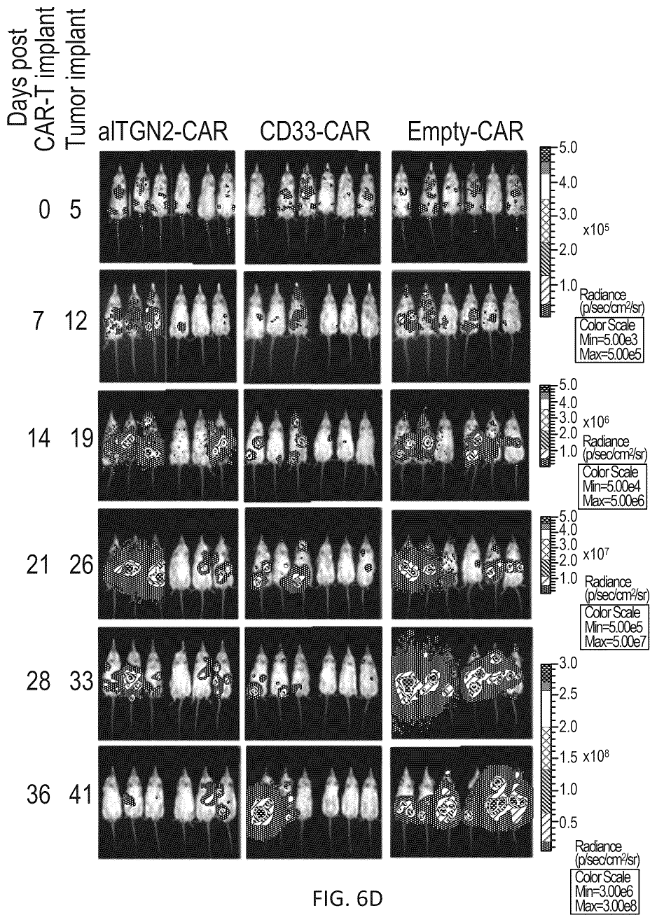

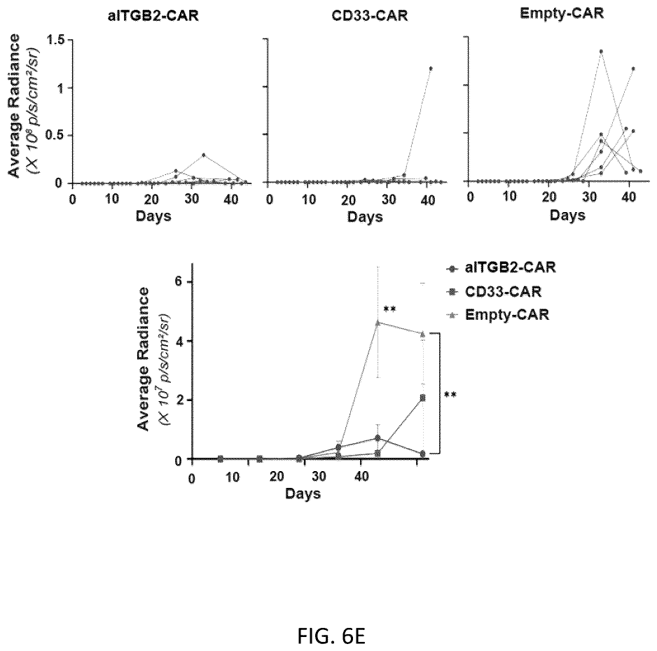

The application figures additionally illustrate development of an antibody to activated Itgβ2 and CAR-T cells generated using the antibody. In brief, FIG. TA-D provides a schematic diagram of surfaceomics as detailed below. FIG. 2A-E illustrates that activated Itgβ2 is a conformationally selective antigen of AML. FIG. 3A-D, further illustrates development of antibody against activated Itgβ2. FIG. 4A-E, shows data relating to development and optimization of CAR-T against AML using the antibody (also referred to as aItgβ2 CAR-T A). FIG. 5A-I provides tdata illustrating the toxicity of aItgβ2 CAR-T. FIG. 6A-E provides survival data from a mouse AML cell tumor model.

Cell Surface Proteomics can be Integrated with Crosslinking Mass Spectrometry (XL-MS) to Probe Structural Information

Cell surface proteins account for just 2% of the total proteome and therefore enrichment of such proteins is desirable for analysis. Because cell surface proteins are primarily N-glycoproteins, we used a glycoxidation approach to install an enrichment handle, biotin, to facilitate their enrichment using Neutravidin beads. Our strategy to identify conformation-selective antigens in cancer involved the further incorporation of cross-linking mass spectrometry (XL-MS) following cell surface protein enrichment. To merge cell surface enrichment with XL-MS, we cross-linked the cells with DSSO or PhoX immediately after harvest to freeze its structural conformation 3D space.

We undertook two different approaches for downstream XL-MS analysis using two separate cross-linkers, namely DSSO and PhoX. These two cross-linkers have a different radius of reactivity and thereby capture complementary sets of cross-linked peptides. DSSO is an MS cleavable cross-linker but does not have any handle for enrichment of cross-linked peptides. The cross-linked peptides being inherently of very low abundance demands low complexity samples for optimal coverage during MS analysis, especially for proteome scale. We thus performed SEC fractionation to simplify the peptide samples. The cross-linked peptides with higher molecular weight elute earlier than regular peptides, primarily in fraction 13 and 14 of the SEC trace. These two fractions were then subjected to high pH fractionation for further simplification of the peptide mixtures which were then analyzed with our MS3 based XL-MS. PhoX on the other hand is non-MS cleavable but has an inbuilt phosphonate handle for enrichment of cross-linked peptides. To further simplify the peptide mixture, immobilized metal affinity chromatography (IMAC) purified peptides were fractionated using SEC column after which the desired fractions were subjected to MS2 analysis. Using these two approaches together, we were able to obtain peptides from the cell surface proteins that provided structural information.

### Structural Surfaceomics Reveals Active Itgβ2 as an AML Target

We profiled cell surfaceome of an AML cell line Nomo1 with our structural surfaceomics approach, which yielded cross-linked peptides, some of which were loop-linked peptides. A number of these cross-linked peptides were from the ItgaL/Itgβ2 heterodimeric protein complex. To determine the legitimacy of these cross-links, we mapped them on to the published crystal structure of Itgβ2 and found agreement with the structure, meaning that the distances between the participating lysine residues were consistent with the radius of reactivity (<20 Å for DSSO) of the cross-linker used. However, 4 cross-links didn't conform to this distance constraint and were of 38.5 Å. These cross-links originated from 2 lysines, each in the I domain of Itgal and the β1 domain of ltgβ2, which in combination formed 4 separate cross-links. This suggested that these two domains are much closer in Nomo1, which is consistent with the active form of Itgβ2.

We further validated the presence of active Itgβ2 in five different AML cell lines with flow cytometry using commercially available M24 antibody, which is known to selectively bind to Itgβ2 active form when present in the heterodimer known as LFA-1. Also, as a negative control, we showed that non-AML cell lines such as BV-173 and Namalwa do not have any active Itgβ2, although they do have total Itgβ2. We also validated the presence of activated Itgβ2 in primary patient AML samples.

### Existing Antibodies Against Active Itgβ2 do not Generate Functional CAR-T Therapy Against AML Cells

In order to target active Itgβ2 for development of CAR-T therapy against AML, we formulated the heavy and light chain variable regions of two known existing antibodies, M24 (Hogg et al, 1985, supra) and AL57 (Shimaoka et al, Proc. Natl. Acad Sci. 2006, supra), into a format expected to bind active Itgβ2 into CAR-T format. For each antibody we tried two different designs in which the V_{H} was followed by the V_{L} and were separated by 3 repeats of a Gly4Ser sequence to allow better folding of the two domains. In the other design we swapped the V_{H} and V_{L} positions. However, neither of the designs exhibited appreciable cytotoxicity against the AML cell line Nomo1 in vitro. We thus sought to develop new antibodies against active Itgβ2 for CAR-T therapy development against AML.

### Phage Display Library Yields Antibodies Selective for Active Itgβ2

Using our phage display antibody engineering platform, we identified six Fabs against recombinant Itgβ2. These Fabs were cloned into a human framework to generate antibodies for flow cytometry-based validation. In order to validate the antibodies, we selected the Jurkat cell line, which is known to have high Itgβ2, but only a minor fraction of which is active. In the presence of Mn²⁺ ions in the staining buffer, all the integrins are activated, thus allowing identification of conformationally selective antibodies. Out of the six antibodies, four were validated by flow cytometry analysis on Jurkat cells. Two of the four bound total Itgβ2 whereas the other two antibodies were specific for the active form of Itgβ2, as demonstrated by the loss of staining in the absence of Mn²⁺ ions in the staining buffer. We also determined the binding affinities of all these antibodies using bioluminescent interferometry (BLI). One antibody (clone 7065) had a K_{D} of 1.56 nM. The second antibody (clone 7341) had a K_{D} of 4.07 nM.

Active Itgβ2 Specific Antibodies can be Reconstructed to scFv for CAR-T Design

Two of the antibodies that were found to be selective against active Itgβ2 namely, antibody clones 7065 and 7341, were reconstructed into scFv format for cloning in a CAR-T backbone. For each antibody, we evaluated two different designs of CAR-T, one with the V_{H} followed by the V_{L} with 3 repeats of a Gly4Ser linker sequence. In the other design, we switched the positions of the V_{H} and V_{L} sequences. The design using antibody 7065 with the V_{L} followed by the V_{H} exhibited higher cytotoxicity (27% greater) at a 1:1 E:T ratio than the cytotoxicity observed using the design with the V_{H} followed by the V_{L}) against Nomo1 in vitro compared to the other designs. However, we also observed issues with CAR-T generation resulting in a low yield of cells. We hypothesized that this was due to fratricide since T cell activation is an important step in CAR-T manufacturing and activated T cells express active Itgβ2. Furthermore, we also observed that even the best performing CAR-T design had moderate killing of the Nomo1 cells. We again suspected that the compromised activity of the CAR-T was due to its prolonged cytotoxic activity during fratricide. Thus, we elected to knockout Itgβ2 in the primary T cells before (in this case 24-hours prior to) transducing them with the CAR-carrying virus. This not only resolved the manufacturing issue of the aItgβ2 CAR-T cells, but also provided efficient cytotoxicity against Nomo1.

We also varied the linker length between the V_{L} and V_{H} regions in scFv fromats of antibody 7065. We employed 1-4 tandem repeats of a Gly4Ser linker sequence and tested them for cytotoxicity against AML cell line Nomo1. Based on the results, we selected two designs: the design with three tandem repeats of the linker sequence and the design with four tandem repeats of the linker sequence.

aItgβ2 CAR-T is Specific Against Active Itgβ2

In order to demonstrate the specificity of aItgβ2 CAR-T against active Itgβ2, we generated an Itgβ2 knock out AML cell line Nomo-1 cell line using CRISPR-Cas9. We then challenged the wild type and knockout Nomo-1 cell lines with aItgβ2 CAR-T. The results showed that aItgβ2 CAR-T was specifically cytotoxic against the wildtype cell line, but not the knockout cell line. This results shows the specificity against the protein Itgβ2, but not necessarily the active form. In order to demonstrate specificity again the active form, we used two B-ALL cell lines, Namalwa and SEM. Namalwa harbors Itgβ2 but not an active form, whereas SEM has a low abundance of active Itgβ2. We challenged these two cell lines with aItgβ2 CAR and found no cytotoxicity against Namalwa and moderate cytotoxicity against SEM. Nomo1 was included as a positive control, for which we observed very efficient killing as expected. This experiment further substantiated the specificity of our aItgβ2 CAR-T against active Itgβ2.

In addition, we also showed that aItgβ2 CAR-T depletes a subpopulation of activated T cells, which harbor active Itgβ2. Notably, our aItgβ2 CAR-T was non-toxic to resting T cells, which have Itgβ2 but not active Itgβ2. The activated T cells were all positive for CD69, thus confirming that the T cells were indeed activated. However, only a subpopulation of T cells was positive for active Itgβ2, which explains the cytotoxicity results. We also observed some active Itgβ2 in resting T cells, which could be due to overnight culture and treatment in vitro; however, activation-time dynamics overnight may not have been sufficient for CAR-T mediated cytotoxicity against them. Further, CD69 is an early activation marker while active Itgβ2 is a late activation marker and needed only for the final immune synapse formation for the delivery of cytotoxic payload.

aItgβ2 CAR is Selectively Cytotoxic to AML Cells

Given the restrictive abundance of Itgβ2 in hematopoietic tissue, we focused on immune cells to underscore the toxicity associated with aItgβ2 CAR-T. We showed using flow cytometry analysis that active Itgβ2 is absent in HSPC, T and B cells. Given the important role of active Itgβ2 in activated granulocytes for migration into tissue and response against infection, its absence in resting myeloid cells is well known, making it a target with a favorable toxicity profile.

We also evaluated the toxicity concerns at CAR-T level using experiments in which we co-cultured aItgβ2 CAR-T with CD34+ HSPCs for 5 hours, followed by clonogenic assay. No cytotoxicity against CD34+ HSPCs was observed. We also co-cultured aItgβ2 CAR-T with with PBMC and again did not observe depletion of T cell or B cells, as evaluated by flow cytometry. However, granulocytes were found to be depleted in this assay. While provide a de facto a positive control for these assays to demonstrate the potency of CAR-T against antigen-bearing cells, toxicity as such against granulocytes is an artifact, given that they are well-known to harbor active Itgβ2 only in activated state when analyzed in vitro, and not the resting state. Thus, it appeared that they were activated in in vitro conditions, which has also been previously reported.

We further demonstrated the presence of active Itgβ2 in vitro using flow cytometry analysis to substantiate that their depletion was an artifact in our assays. In order to address this, we performed a myeloid toxicity assay in vivo in which granulocytes should be in resting state with inactive Itgβ2. Specifically, we generated human immune system (HIS) mice in which we reconstituted the human immune system by injecting human CD34+ cells and allowing the engraft and differentiation into normal human blood cell lineage. We challenged the HIS mice with aItgβ2 CAR-T for 6 days and found no depletion of CD14+ myeloid cells by flow cytometry. Owing to the large variability in CD34+ cells engraftment efficiency and myeloid vs lymphoid population distribution, as determined by flow cytometry analysis using human human CD45 antibody, rigorous quantitative analysis could not be performed. However, in order to bolster our assay results, we included CD33 CAR as a technical positive control, for which we found a substantial decline in ratio of pre- vs post-CAR-T treatment abundance of human CD45+ cells, thus authenticating the success of this assay.

The antibody 7065 employed for generation of aItgβ2 CAR-T was also found to cross-react with mouse Itgβ2 version. This finding therefore provided the opportunity to evaluate the toxicity of aItgβ2 CAR-T in mice against the mouse hematopoietic system. We thus performed complete blood count (CBC) profiling of peripheral blood obtained from HIS mice challenged with aItgβ2 CAR-T. No abnormal counts of any of the hematopoietic cells were identified, thus further supporting the safety profile of aItgβ2 CAR-T.

aItgβ2 CAR is Efficacious Against AML In Vivo

Finally, to demonstrate the efficacy of aItgβ2 CAR-T in vivo, we demonstrated that it successfully depleted the AML cells Nomo1 and THP1 as suggested by the bioluminescence signals. CAR-T treatment also substantially prolonged the survival of the tumor-bearing mice. To further demonstrate efficacy of aItgβ2 CAR-T, we employed two AML PDX mouse models. In both models, we observed pronounced prolongation of survival upon CAR-T treatment. As our PDX did not harbor any marker for non-invasive tumor monitoring, we analyzed peripheral blood samples obtained from the mice at several point time points during the study. In one PDX model, 5 out of 6 mice treated with aItgβ2 CAR-T survived beyond 100 days, as opposed to the negative control “empty” CAR-T treated arms where all the mice were flagged for euthanasia by 70 days. We also performed ultrasound imaging of the spleen, as these PDX were spleen derived, and saw massive spleen enlargement in the mouse that were administered control empty CAR-T, whereas normal spleen size was observed in the mice treated with aItgβ2 CAR-T.

### Summary of Technological Observations

Our structural surfaceomics approach to interrogate the cell surface proteins provides a unique opportunity to uncover cancer antigens that are protein conformation-specific. Notably, such antigens are invisible to stand-alone transcriptome and surface proteomics approaches. Restrictive targeting of such specific protein conformations allows therapeutic targeting of proteins that are otherwise more widely expressed, thereby expanding the scope of therapeutic intervention of cancer or any disease per se. This approach may also be extendible to immunotherapeutic applications for infectious disease as well. Indeed, binding reagent-mediated conformational locking of SARS-CoV-2 spike protein into an inactive mode has been recently reported as a promising therapeutic strategy to combat Covid-19.

Unlike conventional structural biology approaches employing protein crystallization, our structural proteomics strategy is relatively quicker, higher throughput and unbiased. Thus, it can serve as a competing alternative for therapeutic target discovery applications.

As a major focus of this study, we identified active conformation of Itgβ2 as a therapeutic target for treatment of AML and successfully targeted it to develop a CAR-T therapy. Interestingly, Itgβ2 is long known to be widely expressed in hemopoietic tissue and thus to our knowledge has not been previously identified as a cancer target. We now demonstrate that its active form is fairly specific for AML cells. Although activated Itgβ2 is also present on activated T cells and granulocytes, it is absent in their resting state. Granulocytes are well known to harbor activated Itgβ2 for adhesion and migration through the tissues to the site of infection. Thus, the resting cells in peripheral blood would not be visible to our aItgβ2 CAR-T and thus no cytotoxicity should be observed against.

aItgβ2 CAR-T may compromise the ability of a patient to fight infection to some extent. However, this will likely be mitigated in situations in which the site of infection and tumor burden are spatio-temporally separate. This would also hold true for T cells, given that activated T cells are primarily found in tissues. Moreover, active Itgβ2 is a very late marker of activated T cells, which, as noted above, is only required for the final synapse formation. Thus, the CAR-Ts may not have a sufficient time window for cytotoxic action against T cells. In view of the above, aItgβ2 CAR-T is believed to carry a risk of limited toxicity compared to several front runner CAR-T therapies of AML, which are known to lead to severe myeloablation or other off-target effects.

Expression of Itgβ2 and its activation is an important step in CAR-T manufacturing and as noted above initially posed a challenge. However, we were able to successfully resolve this issue by knocking out Itgβ2 before CAR transduction using CRISPR-Cas9.

Not all AML patients have high degree of active Itgβ2, which is not unexpected in light of the known heterogeneity of AML surface marker expression. Follow up affinity maturation, e.g., using a yeast or phage display platform or using other revised CAR designs, to provide mor epotency against activated Itgβ2, may address situations in which active Itgβ2 is only present in AML at low densities. Regardless, given the complementary or inverse expression pattern of Itgβ2 against the other targets in AML patients, aItgβ2 CAR-T may be a good addition to the currently limited regime of AML treatment in the clinic. The antibody binding domain in the the CAR-T design of the present disclosure is from a human framework and is thus ready for therapeutic applications with minimal immunogenicity concerns. Lastly, the current study also serves as an excellent proof of concept to demonstrate that a protein conformation-based cancer antigen can be mined with an unbiased structural proteomics profiling of cancer cell surfacse and can also be targeted for immunotherapeutic applications.

### Materials and Methods

Cell lines, PDX and patient samples. Nomo1, THP1, HL60, MV411, Jurkat, SEM, and Namalwa cell lines were all grown in RPMI-1640 with 20% FBS and 100 U/ml Penicillin-Streptomycin (pen-strep) in 5% CO₂ at 37° C. All the AML PDX were procured from Public Repository for Xenografts (PRoXe) at Dana-Farber Cancer Center. Primary AML samples were obtained from HMTB and Helen Diller Cancer Research Center Tissue Bank.

Generation of Itgβ2 knockout cell line: Knockout cell lines of primary T cells were generated using invitro nucleofection of Cas9 ribonuclease protein complex. Briefly, 2 ul each of sgRNA and recombinant Cas9 protein (UC Berkeley Cell Culture Facility) were incubated at 37° C. for 15 minutes. One million cells were washed once with PBS (500 RCF for 5 min at RT) and resuspended in a mixture of 16.4 ul SF cell line solution and 3.6 μl supplemental solution-1. The sgRNA and Cas9 ribonuclease protein complex from the incubation was then mixed with the cell suspension. 20 ul of the suspension was aliquoted to a cuvette and nucleofected using Lonza with the inbuilt program DS-137 for cell lines and EO-115 for primary T cells. The cells were then allowed to briefly rest at room temperature and added to fresh media pre-warmed at 37° C.

Cross-linking and Cell surface labelling: 2.4×10⁹ cells (in batches of 6×10⁸) were harvested and washed (300 RCF for 5 min) three times with PBS each time to remove the amine-containing components of the media and then resuspended in PBS. The amine reactive cross-linker DSSO pre-dissolved in DMSO was added to the cells at a final concentration of 10 mM and incubated at RT for 45 minutes. The cross-linking step was followed by biotinylation of the cell surface proteins using glycoxidation chemistry of the N-linked glycosylation-site. Briefly, the cells were then washed with PBS three times and treated with 1.6 mM sodium metaperiodate (VWR, 13798-22) for 20 minutes at 4° C. for oxidation of the N-linked sugar residues. The cells were again washed twice with PBS and treated with 10 mM Aniline (Sigma-Aldrich, 242284) and 1 mM biocytin hydrazide (Biotium, 90060) for 90 minutes at 4° C., for installation of biotin on the oxidized sugar residues. The cells were again washed three times to remove excess biotinylating reagents, snap frozen in liquid nitrogen, and stored at −80° C. until further processing. All the incubation steps were carried out in an end-to-end rotor for gentle mixing during the reactions.

Proteomics sample preparation: The frozen cell pellets were thawed in ice and were resuspended in 1 ml RIPA lysis buffer with Halt protease inhibitor and 1 mM EDTA. The cell suspension was then sonicated to lyse the cells followed by incubation in ice for 10 minutes with intermittent vortexing every 2-3 minutes. The lysate was then centrifuged at 17000 RCF for 10 minutes at 4° C. to obtain the clarified supernatant containing the biotinylated cell surface proteins. This clear supernatant was added to the 0.5 ml of Neutravidin beads prewashed and equilibrated with RIPA lysis buffer+1 mM EDTA abd incubated at 4° C. for 2 hours, after which the beads were removed for further processing. To remove non-specifically bound proteins, the beads were washed extensively using a vacuum manifold, consecutively with 50 ml RIPA lysis buffer+1 mM EDTA, 50 ml PBS+1 M NaCl and 50 ml 2 M Urea+50 mM Ammonium Bicarbonate. The beads bound with biotinylated cell surface proteins were resuspended in 50 mM Tris (pH-8.5)+4 M urea+10 mM TCEP and 20 mm IAA. 10 ug Trypsin-LysC mix was added to this mixture to allow on-bead digestion of the bound proteins for simultaneous reduction and alkylation of cysteines residues at RT in end-to-end rotor. After 2 hours, the mixture was diluted to 1.5 M urea using 50 mM tris (pH-8.5), whereby Trypsin also is activated. Protease digestion was conducted overnight (16-20 hours). The solution was centrifuged to pellet the beads and the supernatant containing the tryptic peptides was transferred to a fresh tube and acidified with 0.5% Trifluoroacetic acid (TFA). The peptides were then desalted using a SOLA HRP Column and eluted with 50% acetonitrile (ACN)+0.1% formic acid (FA). Finally, the peptides were dried down in speedvac.

IMAC purification: Dry peptides were reconstituted in 80% CAN+0.1% TFA. Meanwhile, Superflow Ni-NTA beads were stripped using EDTA and reloaded with FeCl₃ in apolyprep chromatography column. Fe-loaded beads were transferred to C18 nest tips and incubated for 4-6 minutes with intermittent mixing with the reconstituted peptides to allow specific binding of the PhoX (cross-linker with IMAC handle) bearing peptides. The beads were then rigorously washed with 0.5% FA to remove the unbound or the non-specifically bound peptides. The bound peptides were then eluted with 0.5 M Potassium Phosphate (pH—7.4). The peptides eluted from the beads is again bound to the C18 bed of the nest tips, which is again washed three times e with 0.5% FA to remove the Potassium Phosphate, and finally eluted with 50% ACN+0.1% FA and dried down in speed vac.

Size Exclusion Chromatography: Size based fractionation of the peptides was performed using a Superdex peptide column and HPLC. The peptides were reconstituted in the mobile phase constituting 30% ACN+0.10% TFA and loaded onto the column. The run time was 90 minutes at a flow rate of 50 μl per minute and 45 fractions (2 minutes per fraction) were collected in total.

LC-MS analysis: For each run of DSSO cross-linked samples, 1 μg of peptides from each high pH fractions was loaded on to an EASY-Spray™ HPLC Column for a LC system. The peptides were separated over a period of 4 hours using a gradient buffer A (0.1% FA) and buffer B (100% ACN+0.1% FA).

For each run of PhoX cross-linked samples, 1 μg from selected SEC fractions were loaded on to the column. The peptides were separated over a period of 4 hour gradient using buffer A (0.1% FA) and buffer B (100% ACN+0.1% FA). The peptides eluting from the column were analyzed using Tims-TOF pro instrument.

### MS Data Analysis:

Flow cytometry: Immunostaining of cells was performed as per the instructions from the antibody vendor unless stated otherwise. Briefly, 1 million cells were resuspended in 100 μl of FACS buffer (PBS+2% FBS) with 1 ug antibody added to it. The cells were incubated at 4° C. for 10-15 minutes and then washed three times with FACS buffer. For staining active form of Itgβ2, antibody incubation step was performed at 37° C. for 1 hour. In the case of staining primary AML cells for activated Itgβ2, the FACS buffer was RPMI-1640+5% FBS+2% BSA+50 μg/ml DNase-I. For all other primary cell staining, the FACS buffer was D-PBS+5% FBS+2% BSA+5 mM EDTA+50 μg/ml DNase-I.

Phage display selections: A synthetic, phage-displayed antibody library (PMID: 23219464) was selected for binding to either Integrin beta-2/Integrin alpha-M (R and D 4047-AM, Antibody #7062, 7 #063, #7065) or Integrin beta-2/Integrin alpha-L (R and D 3868-AV, Antibody #7060, #7341) recombinant protein complexes. Briefly, Integrin-beta-2 recombinant protein complexes were immobilized on Maxisorp Immuno plates (ThermoFisher, #12-565-135) and used for positive binding selections with library phage pools that were first exposed to neutravidin coated wells to deplete nonspecific binders. After four rounds of binding selections, clonal phage was prepared and evaluated by phage ELISA and sequencing as described (PMID: 23219464).

Antibody production: Antibodies were produced using the human Expi293 expression system (Thermofisher). Expi293 cells at 2 ml volume were transiently transfected with construct DNA using FectoPro transfection reagent (Polyplus Transfection). Following a 5-day expression period, antibodies were purified using rProteinA Sepharasoe (GE Healthcare) and stored in phosphate buffer (50 mM NaH2PO4, 75 mM Na2HPO4, 100 mM H3PO4, 154 mM NaCl).

Bio-Layer Interferometry (BLI) binding assays: The binding of human Integrin beta-2 antibodies was tested against three different Integrin beta-2 complexes including Integrin beta-2/Integrin alpha-M (R and D 4047-AM), Integrin beta-2/Integrin alpha-X (R and D 5755-AX), and Integrin beta-2/Integrin alpha-L (R and D 3868-AV). To determine the binding kinetic parameters of the antibodies, BLI experiments were performed on an Octet HTX instrument (Sartorius) at 1000 rpm and 25° C. All proteins were diluted in an assay buffer (PBS, 1% BSA, 0.05% Tween 20). Test and control antibodies at a concentration of 2 μg/ml were first captured on AHQ biosensors to achieve the binding signals of 0.8-1.3 nm. Unoccupied Fc-binding sites on the antibody-coated sensors were subsequently quenched by 20 μg/mL of the Fc protein. After equilibration with the assay buffer, the biosensors were then dipped for 600 s into wells containing 5-fold serial dilution of Integrin-beta 2 complexes (association phase), followed by a transfer back into an assay buffer for additional 600 s (dissociation phase). Assay buffer alone served as a negative control. Binding response data were reference subtracted and were globally fitted with 1:1 binding model using ForteBio's Octet Systems software 9.0.

Non-specific Elisa panel: The ELISA protocol to assess interactions of the antibodies with unrelated macromolecules was adapted from Jain et al (PMID: 28096333). The tested antigens included Cardiolipin (50 μg/mL, Sigma C0563), KLH (5 μg/mL, Sigma H8283), LPS (10 μg/mL, InvivoGen tlrl-eblps), ssDNA (1 μg/mL, Sigma D8899), dsDNA (1 μg/mL, Sigma D4522), and Insulin (5 μg/mL, Sigma 19278). In addition, the binding of each antibody was also tested against empty wells (BSA only control) and wells containing goat anti-human Fe antibody (positive control, 1 μg/ml, Jackson 109-005-098). The antigens were coated at 30 uL per well in 384-well Maxisorp plates and incubated at 4° C. overnight. Plates were blocked with 0.5% bovine serum albumin (BSA) for 1 hour at room temperature and washed with PBS+0.05% Tween20. The antibodies were added at 100 nM and allowed to bind for 60 min at room temperature. Plates were washed with PBS+0.05% Tween20 and binding was detected with anti-kappa HRP antibody (1:5000, Southern Biotech #2060-05) and developed with the TMB substrate (KPL (Mandel) KP-50-76-03).

Plasmid constructs: All plasmid constructs were generated using NEBuilder® HiFi DNA Assembly Master Mix as per the vendor's instructions with some modifications. The DNA fragments containing the binder (scFv) sequence along with the 40 bp vector compatible flanking region for gibson assembly was procured from TWIST. Meanwhile, the target CAR plasmid backbone was linearized with BamHI-HF and cleaned up using Zymo Research DNA purification kit. 10 ng of linearized vector and 5 ng of the DNA fragment (insert) was used to set 10 ul of gibson assembly reaction. This reaction mixture was then transformed into stbl3 competent E. coli cells and the colonies obtained were screened for the positive clone.

Primary T cell isolation: Primary T cells were isolated from leukopaks obtained from Stem Cell Technologies. CD8 and CD4 cells were isolated separately using their EasySep™ Human CD8+/CD4+ T Cell Isolation Kit as per manufacturer's instructions. Briefly, all the unwanted cells were labelled with magnet conjugated antibody cocktail which is separated using their EasySep magnetic stand leaving CD4 or CD8 cell in suspension. This negative selection approach results in isolation of untouched CD8 or CD4 T cells and stored frozen with 10% DMSO.

CAR T generation: T cells were thawed and grown in T cell media constituting Optmizer CTS media+CTS supplement+5% Human AB Serum+Penicillin/Streptamycin+glutamax. Recombinant human IL7 and IL15 was freshly added to the cells every 2-3 days. For manufacturing CAR-T cells, primary T cells (CD4 or CD8) were thawed and cultured overnight. The cells were then nucleofected with ribonuclease complex of Itgβ2 sgRNA and Cas9 using P3 Primary Cell 4D-Nucleofector™ X Kit S with its inbuilt program EO-115. The cells were then stimulated with 20 μl of CD3/CD28 Dynabeads (11131-D; Thermo Fisher Scientific) per million cells. Meanwhile, lentivirus carrying the CAR expression cassette was added to the cells the day after adding the stimulation beads. The virus was withdrawn from the culture after 24 followed by 2-3 rounds of PBS wash using centrifugation at 300 RCF for 5 minutes. Assuming bead stimulation as day 0, beads were withdrawn on day 4 using magnetic rack and the T cells. On day 6 or 7, the cells were MACS sorted for the CAR positive cells using c-myc tag of the CAR constructs as a handle. The CAT-T cells were used for in vitro and in vivo studies within day 10-14 of the manufacturing process.

T cell activation assay: PBMC cells were treated with 3 μM ionomycin+25 ng/ml LPS+100 U/ml IL-2 and cultured overnight in C02 incubator. The cells were then co-stained with CD3 and CD69 and analyzed with flowcytometry. CD3 was used to gate on T cells and CD69 was used as a T cell activation marker.

In vitro cytotoxicity assay: The AML cell lines used for in vitro cytotoxicity analysis were engineered to stably express luciferase using lentiviral transduction. The cell lines were co-cultured overnight with CAR-T cells in various ratios in a 96 well white plate. 150 μg/mL of d-luciferin (LUCK-IG; Gold Biotechnology) was then added to each well and incubated for 5 minutes at RT, after which the plate is read for luciferase signal using GloMax Explorer Plate Reader (Promega). For each ratio (CAR-T: Tumor), the bioluminescence reading from the tumor cells co-cultured with untransduced T cells were considered 100% viable and thus used for normalization.

Clonogenic assay: 1,000 CD34+ cells from healthy donor mobilized peripheral blood were co-incubated with aItgβ2 CAR, CD33 CAR, Empty CAR T cells, untransduced T cells or medium only (IMDM, 2% FBS, penicillin/streptomycin) at an E:T ratio of 1:1 for 5 hours in V-bottom 96 well plates and then plated in triplicate in methylcellulose-based medium supplemented with recombinant cytokines (MethoCult H4434 Classic, STEMCELL Technologies). After 13-14 days, colonies were classified and counted as CFU-GEMM, GM, G, M, or BFU-E. Pictures were taken with a Keyence microscope using 10×, brightfield, color mode.

Mice experiments: All the mice experiments were 6-8 weeks old and obtained from either Jackson laboratory (NSG-SMG3) or from in-house (NSG) bred stocks of Pre-Clinical Therapeutics Core of UCSF. Each mice were injected with 1 million AML cell lines or 2 million PDX AML lines intravenously through tail vein. In case of PDX, the mice were irradiated 4-6 hours prior to injection. 4-5 days later, the mice were treated with 4-5 million CAR-T cells at 1:1 ratio of CD4 and CD8. Tumor burden in case of cell lines (luciferased lines) was assessed using bioiluminecense imaging with Xenogen In Vivo Imaging System (Caliper Life Sciences). In case of PDX, using flowcytometry analysis of blood draws and spleen size determination with ultrasonography was used as a readout for tumor burden. All the mice experiments were conducted in accordance with UCSF Institutional Animal Care and Usage Committee.

HIS mice generation: All the mice used for HIS mice generation were of NSG-SMG3 strain and obtained from Jackson laboratory. Each mice was treated with busulfan followed by injection with 70K CD34 cells intravenously through tail vein. The human CD34 cells enriched blood samples were obtained from Bone Marrow and Transplantation Laboratory of UCSF which were MACS sorted using CD34 MicroBead Kit prior to injection in mice. The blood draw of these mice were analyzed using flowcytometry 8-10 weeks post CD34 cells injection, to determine the engraftment efficiency using Human CD45+ cells as a read out.

Statistical analysis: All the statistical analysis were done using GrpahPad Prism unless stated otherwise. The data have been represented as ±mean and pValue <0.05 were considered statistically significant.

### Illustrative Polypeptide Sequences:

Uniprot P05107-1 amino acid sequence
SEQ ID NO:1
MLGLRPPLLA LVGLLSLGCV LSQECTKFKV SSCRECIESG PGCTWCQKLN
FTGPGDPDSI RCDTRPQLLM RGCAADDIMD PTSLAETQED HNGGQKQLSP
QKVTLYLRPG QAAAFNVTFR RAKGYPIDLY YLMDLSYSML DDLRNVKKLG
GDLLRALNEI TESGRIGFGS FVDKTVLPFV NTHPDKLRNP CPNKEKECQP
PFAFRHVLKL TNNSNQFQTE VGKQLISGNL DAPEGGLDAM MQVAACPEEI
GWRNVTRLLV FATDDGFHFA GDGKLGAILT PNDGRCHLED NLYKRSNEFD
YPSVGQLAHK LAENNIQPIF AVTSRMVKTY EKLTEIIPKS AVGELSEDSS
NVVQLIKNAY NKLSSRVFLD HNALPDTLKV TYDSFCSNGV THRNQPRGDC
DGVQINVPIT FQVKVTATEC IQEQSFVIRA LGFTDIVTVQ VLPQCECRCR
DQSRDRSLCH GKGFLECGIC RCDTGYIGKN CECQTQGRSS QELEGSCRKD
NNSIICSGLG DCVCGQCLCH TSDVPGKLIY GQYCECDTIN CERYNGQVCG
GPGRGLCFCG KCRCHPGFEG SACQCERTTE GCLNPRRVEC SGRGRCRCNV
CECHSGYQLP LCQECPGCPS PCGKYISCAE CLKFEKGPFG KNCSAACPGL
QLSNNPVKGR TCKERDSEGC WVAYTLEQQD GMDRYLIYVD ESRECVAGPN
IAAIVGGTVA GIVLIGILLL VIWKALIHLS DLREYRRFEK EKLKSQWNND
NPLFKSATTT VMNPKFAES
Antibody D3-7065 heavy chain variable region; CDRs are underlined
SEQ ID NO: 2
EVQLVESGGGLVQPGGSLRLSCAASGFTISYYYMHWVRQAPGKGLEWVASISSSSG
YTYYADSVKGRFTISADTSKNTAYLQMNSLRAEDTAVYYCARGAMDYWGQGTLV
TVSS
Antibody D3-7065 light chain variable region; CDRs are underlined
SEQ ID NO: 3
DDIQMTQSPSSLSASVGDRVTITCRASQSVSSAVAWYQQKPGKAPKLLIYSASSLYSG
VPSRFSGSRSGTDFTLTISSLQPEDFATYYCQQFSSGSWAPITFGQGTKVEIK
HCDR1:
(SEQ ID NO: 13)
ISYYYM
HCDR2:
(SEQ ID NO: 14)
SISSSSGYTY
HCDR3:
GAM
LCDR1:
(SEQ ID NO: 16)
SVSSA
LCDR2:
(SEQ ID NO: 17)
SASSLYS
LCDR3:
(SEQ ID NO: 18)
FSSGSWAPI
Gly4Ser Linkers sequences:
Linker for C1 and D1:
(SEQ ID NO: 19)
GGGGS
Linker for C2 and D2:
(SEQ ID NO: 20)
GGGGSGGGGS
Linker for C3 and D3:
(SEQ ID N O: 21)
GGGGSGGGGSGGGGS
Linker for C4 and D4:
(SEQ ID NO: 22)
GGGGSGGGGSGGGGSGGGGS
C1-7065-H-L orientation C1 linker
SEQ ID NO: 4
EVQLVESGGGLVQPGGSLRLSCAASGFTISYYYMHWVRQAPGKGLEWVASISSSSG
YTYYADSVKGRFTISADTSKNTAYLQMNSLRAEDTAVYYCARGAMDYWGQGTLV
TVSSGGGGSDIQMTQSPSSLSASVGDRVTITCRASQSVSSAVAWYQQKPGKAPKLLI
YSASSLYSGVPSRFSGSRSGTDFTLTISSLQPEDFATYYCQQFSSGSWAPITFGQGTKV
EIK
C2-7065-H-L orientation C2 linker
SEQ ID NO: 5
EVQLVESGGGLVQPGGSLRLSCAASGFTISYYYMHWVRQAPGKGLEWVASISSSSG
YTYYADSVKGRFTISADTSKNTAYLQMNSLRAEDTAVYYCARGAMDYWGQGTLV
TVSSGGGGSGGGGSDIQMTQSPSSLSASVGDRVTITCRASQSVSSAVAWYQQKPGKA
PKLLIYSASSLYSGVPSRFSGSRSGTDFTLTISSLQPEDFATYYCQQFSSGSWAPITFGQ
GTKVEIK
C3-7065-H-L orientation C3 linker
SEQ ID NO: 6
EVQLVESGGGLVQPGGSLRLSCAASGFTISYYYMHWVRQAPGKGLEWVASISSSSG
YTYYADSVKGRFTISADTSKNTAYLQMNSLRAEDTAVYYCARGAMDYWGQGTLV
TVSSGGGGSGGGGSGGGGSDIQMTQSPSSLSASVGDRVTITCRASQSVSSAVAWYQQ
KPGKAPKLLIYSASSLYSGVPSRFSGSRSGTDFTLTISSLQPEDFATYYCQQFSSGSWA
PITFGQGTKVEIK
C4-7065-H-L orientation C4 linker
SEQ ID NO: 7
EVQLVESGGGLVQPGGSLRLSCAASGFTISYYYMHWVRQAPGKGLEWVASISSSSG
YTYYADSVKGRFTISADTSKNTAYLQMNSLRAEDTAVYYCARGAMDYWGQGTLV
TVSSGGGGSGGGGSGGGGSGGGGSDIQMTQSPSSLSASVGDRVTITCRASQSVSSAV
AWYQQKPGKAPKLLIYSASSLYSGVPSRFSGSRSGTDFTLTISSLQPEDFATYYCQQF
SSGSWAPITFGQGTKVEIK
D1-7065-L-H orientation D1 linker
SEQ ID NO: 8
DIQMTQSPSSLSASVGDRVTITCRASQSVSSAVAWYQQKPGKAPKLLIYSASSLYSGV
PSRFSGSRSGTDFTLTISSLQPEDFATYYCQQFSSGSWAPITFGQGTKVEIKGGGGSEV
QLVESGGGLVQPGGSLRLSCAASGFTISYYYMHWVRQAPGKGLEWVASISSSSGYT
YYADSVKGRFTISADTSKNTAYLQMNSLRAEDTAVYYCARGAMDYWGQGTLVTVS
S
D2-7065-L-H orientation D2 linker
SEQ ID NO: 9
DIQMTQSPSSLSASVGDRVTITCRASQSVSSAVAWYQQKPGKAPKLLIYSASSLYSGV
PSRFSGSRSGTDFTLTISSLQPEDFATYYCQQFSSGSWAPITFGQGTKVEIKGGGGSGG
GGSEVQLVESGGGLVQPGGSLRLSCAASGFTISYYYMHWVRQAPGKGLEWVASISS
SSGYTYYADSVKGRFTISADTSKNTAYLQMNSLRAEDTAVYYCARGAMDYWGQGT
LVTVSS
D3-7065-L-H orientation D3 linker
SEQ ID NO: 10
DIQMTQSPSSLSASVGDRVTITCRASQSVSSAVAWYQQKPGKAPKLLIYSASSLYSGV
PSRFSGSRSGTDFTLTISSLQPEDFATYYCQQFSSGSWAPITFGQGTKVEIKGGGGSGG
GGSGGGGSEVQLVESGGGLVQPGGSLRLSCAASGFTISYYYMHWVRQAPGKGLEW
VASISSSSGYTYYADSVKGRFTISADTSKNTAYLQMNSLRAEDTAVYYCARGAMDY
WGQGTLVTVSS
D4-7065-L-H orientation D4 linker
SEQ ID NO: 11
DIQMTQSPSSLSASVGDRVTITCRASQSVSSAVAWYQQKPGKAPKLLIYSASSLYSGV
PSRFSGSRSGTDFTLTISSLQPEDFATYYCQQFSSGSWAPITFGQGTKVEIKGGGGSGG
GGSGGGGSGGGGSEVQLVESGGGLVQPGGSLRLSCAASGFTISYYYMHWVRQAPG
KGLEWVASISSSSGYTYYADSVKGRFTISADTSKNTAYLQMNSLRAEDTAVYYCAR
GAMDYWGQGTLVTVSS

1.-43. (canceled)

44. A method of treating a subject having a cancer comprising cells that express activated integrin beta-2 (Itgβ2), the method comprising administering an antibody that specifically binds activated Itgβ2 to the subject, wherein the antibody is linked to a therapeutic moiety and comprises an Itgβ2 binding domain comprising:
a heavy chain variable region (V_{H}) comprising an HCDR1 sequence comprising ISYYYM (SEQ ID NO:13), an HCDR2 sequence comprising SISSSSGYTY (SEQ ID NO:14); and an HCDR3 sequence comprising GAM; and
a light chain variable region (V_{L}) comprising an LCDR1 sequence comprising SVSSA (SEQ ID NO:16, an LCDR2 sequence comprising SASSLYS (SEQ ID NO:17); and an LCDR3 sequence comprising FSSGSWAPI (SEQ ID NO:18).

45. The method of claim 44, wherein the V_{H} comprises an amino acid sequence having at least 95% identity to SEQ ID NO:2 and the V_{L} comprises an amino acid sequence having at least 95% identity to SEQ ID NO:3.

46. The method of claim 45, wherein the V_{H} comprises amino acid sequence SEQ ID NO:2 and the V_{L} comprises amino acid sequence SEQ ID NO:3.

47. The method of claim 44, wherein the antibody comprises a single chain Fv (scFv), a bispecific antibody, or a multispecific antibody.

48. The method of claim 44, wherein the antibody comprises amino acid sequence SEQ ID NO:10 or amino acid sequence SEQ ID NO:11.

49. The method of claim 44, wherein the cancer is acute myeloid leukemia.

50. A method of treating a subject having a cancer comprising cells that express activated Itgβ2, the method comprising administering a plurality of immune effector cells to the subject, wherein the immune effector cells express a chimeric antigen receptor (CAR) comprising an antigen binding domain, an intracellular domain, and a cytoplasmic signaling domain, wherein the antigen binding domain comprises:
a heavy chain variable region (V_{H}) comprising an HCDR1 sequence comprising ISYYYM (SEQ ID NO:13), an HCDR2 sequence comprising SISSSSGYTY (SEQ ID NO:14); and an HCDR3 sequence comprising GAM; and
a light chain variable region (V_{L}) comprising an LCDR1 sequence comprising SVSSA (SEQ ID NO:16, an LCDR2 sequence comprising SASSLYS (SEQ ID NO:17); and an LCDR3 sequence comprising FSSGSWAPI (SEQ ID NO:18).

51. The method of claim 50, wherein the the V_{H} comprises an amino acid sequence having at least 95% identity to SEQ ID NO:2 and the V_{L} comprises an amino acid sequence having at least 95% identity to SEQ ID NO:3; or the V_{H} comprises amino acid sequence SEQ ID NO:2 and the V_{L} comprises amino acid sequence SEQ ID NO:3.

52. The method of claim 50, wherein the V_{L} region of the scFv is N-terminal to the V_{H} region.

53. The method of claim 50, wherein the antigen binding domain comprises amino acid sequence SEQ ID NO:10 or SEQ ID NO:11.

54. The method of claim 50, wherein the immune effector cells are T lymphocytes or a natural killer (NK) cells.

55. The method of claim 50, wherein the plurality of immune effector cells comprises allogeneic cells.

56. The method of claim 50, wherein the plurality of immune effector cells comprises autologous cells.

57. The method of claim 50, wherein the cancer is acute myeloid leukemia.

58. A plurality of immune effector cells expressing a CAR comprising an antigen binding domain, an intracellular domain, and a cytoplasmic signaling domain, wherein the antigen binding domain comprises an scFV comprising a V_{H} comprising an HCDR1 sequence comprising ISYYYM (SEQ ID NO:13), an HCDR2 sequence comprising SISSSSGYTY (SEQ ID NO:14); and an HCDR3 sequence comprising GAM; and a V_{L} comprising an LCDR1 sequence comprising SVSSA (SEQ ID NO:16), an LCDR2 sequence comprising SASSLYS (SEQ ID NO:17); and an LCDR3 sequence comprising FSSGSWAPI (SEQ ID NO:18), wherein the V_{L} region of the scFv is N-terminal to the V_{H} region.

59. The plurality of immune effector cells of claim 58, wherein the antigen binding domain comprises a V_{H} comprising an amino acid sequence having at least 95% identity to SEQ ID NO:2 and the V_{L} comprises an amino acid sequence having at least 95% identity to SEQ ID NO:3; or the V_{H} comprises amino acid sequence SEQ ID NO:2 and the V_{L} comprises amino acid sequence SEQ ID NO:3.

60. The plurality of immune effector cells of claim 58, wherein the antigen binding domain comprises amino acid sequence SEQ ID NO:10 or SEQ ID NO:11.

61. The plurality of immune effector cells of claim 58, wherein the immune effector cells comprise T lymphocytes or NK cells.

62. The plurality of immune effector cells of claim 58, wherein the immune effector cells comprise allogeneic cells.

63. The plurality of immune effector cells of claim 58, wherein the immune effector cells comprise autologous cells.
